(19) United States
(12) Patent Application Publication
(10) Pub. No.: US 2012/0014883 A1
(43) Pub. Date: Jan. 19, 2012

(54) Compositions Comprising Derivatives Of Essential Oil Compounds And Use In Personal Care Products

(76) Inventors: Douglas Craig Scott, Loveland, OH (US); Lowell Alan Sanker, Montgomery, OH (US); Robert Leslie Swaine, JR., Glendale, OH (US)

(21) Appl. No.: 13/185,522

(22) Filed: Jul. 19, 2011

Related U.S. Application Data
(60) Provisional application No. 61/365,418, filed on Jul. 19, 2010.

(51) Int. Cl. A61K 36/54; A61K 36/45; A61K 8/21; A61K 8/97; A61K 8/33; A61K 8/55; A61K 31/216; A61K 31/225; A61P 31/02; A61Q 5/02; A61Q 11/00; A61K 8/22; A61K 36/534; A61K 31/352

(52) U.S. Cl. 424/52; 424/747; 424/739; 424/725; 424/58; 424/49; 514/456; 514/529; 514/548; 424/53; 424/57

(57) ABSTRACT

Compositions containing one or more derivatives of essential oil compounds for use in personal care compositions, such as compositions for oral, throat and skin care are disclosed. These derivatives include acetals of parent essential oil aldehydes and ketones; esters or ethers of parent essential oil alcohols and phenolics; and esters of parent essential oil acids. Examples of parent essential oil aldehydes and ketones include citral, cinnamic aldehyde, ρ-anisaldehyde, vanillin, ethyl vanillin, heliotropin, carvone, and menthone. Examples of parent essential oil alcohols and phenolics include thymol, eugenol, isoeugenol, dihydroeugenol, carvacrol, carveol, geraniol, nerol, vanillyl alcohol, heliotropyl alcohol, ρ-anisyl alcohol, cinnamyl alcohol and β-ionol. Examples of parent essential oil acids include ρ-anisic acid, cinnamic acid, vanillic acid and geranic acid. The present compositions comprising essential oil derivatives are useful as base flavor or base perfume for incorporation into personal care products and to provide other benefits including antimicrobial efficacy. Optionally the compositions will contain additional antimicrobially- or anti-inflammatory-effective components including those also derived from plant essential oils or synthetic versions thereof.

### CROSS REFERENCE TO RELATED APPLICATIONS

This application claims the benefit of U.S. Provisional Application Ser. No. 61/365,418 filed on Jul. 19, 2010.

### TECHNICAL FIELD

The present invention relates to compositions containing one or more chemical derivatives of plant essential oil constituent compounds and their use in personal care products, such as oral, throat and nasal care products to provide benefits including antimicrobial activity as well as a pleasing flavor and aroma that enhances consumer acceptability of the finished products.

### BACKGROUND OF THE INVENTION

Oral care products such as dentifrice and mouthrinse are routinely used by consumers as part of their oral care hygiene regimens to provide both therapeutic and cosmetic hygiene benefits. Therapeutic benefits include caries prevention which is typically delivered through the use of various fluoride salts; gingivitis prevention by the use of an antimicrobial agent such as triclosan, stannous fluoride, or essential oils; or hypersensitivity control through the use of ingredients such as stannous fluoride, strontium chloride or potassium nitrate. Hygiene and cosmetic benefits provided by oral care products include the control of plaque and calculus formation, removal and prevention of tooth stain, tooth whitening, breath freshening, and overall improvements in mouth feel impression which can be broadly characterized as mouth feel aesthetics. Calculus and plaque along with behavioral and environmental factors lead to formation of dental stains, significantly affecting the aesthetic appearance of teeth. Behavioral and environmental factors that contribute to teeth staining propensity include regular use of coffee, tea, cola or tobacco products, and also the use of certain oral products containing ingredients that promote staining, such as chlorhexidine and metal salts.

Dental plaque is a mixed matrix of bacteria, epithelial cells, leukocytes, macrophages and other oral exudates. Bacteria comprise approximately three-quarters of the plaque matrix. Any given sample of dental plaque could contain as many as 400 different varieties of microorganisms. This mix includes both aerobic and anaerobic bacteria, fungi, and protozoa. Viruses have also been found in samples of dental plaque. This matrix of organisms and oral exudates continues expanding and coalesces with other plaque growths situated nearby. The bacteria synthesize levans and glucans from sugars found in the oral cavity providing energy for the microorganisms. These glucans, levans, and microorganisms form an adhesive skeleton for the continued proliferation of plaque into what is also referred to as a biofilm, which is tenaciously adherent and difficult to remove. Mineralized dental plaque biofilms deposit on the surfaces of the teeth at the gingival margin and mature to what is referred to as calculus or tartar. As the mature calculus develops, it becomes visibly white or yellowish in color unless stained or discolored by some extraneous agent and becoming unsightly and aesthetically undesirable.

The failure to retard or stop the proliferation of plaque is detrimental to oral health, leading to dental caries, gingival inflammation, periodontal disease, and ultimately tooth loss. It is widely recognized that dental plaque bacteria, growing in the area where the teeth and gingival tissues meet, cause an inflammation of the gingiva called “gingivitis”. This is characterized by swollen, edematous gingiva (“gums”) which are reddened and bleed easily. If plaque removal is inadequate, gingivitis may progress to “periodontitis” or periodontal disease in many individuals. Periodontitis generally is characterized by a chronic inflammation of the tissues around the teeth, which leads to a resorption of supporting bone. Periodontal disease is the leading cause of tooth loss among adults. Dental caries (cavities) are also bacteria-mediated, with Streptococcus mutans believed to be the principal etiologic agent.

Prevention and removal of dental plaque have long been the focus of development, with the ultimate goal of inhibiting caries, calculus, gingivitis and periodontal diseases. While plaque removal can be accomplished to a certain extent by mechanical means such as by brushing the teeth particularly in conjuncton with abrasive compositions, brushing alone is not sufficient to effectively remove substantially all of the dental plaque that has formed on the teeth or prevent the formation or regrowth of plaque. To complement mechanical means of plaque control, chemical methods using antimicrobials have been proposed.

Among the many antimicrobial agents that have been demonstrated to be effective for use in the oral cavity include chlorhexidine; benzalkonium chloride; cetylpyridinium chloride; triclosan; metal ions such as stannous, zinc and copper; and essential oils. However, many of these oral antimicrobials have the disadvantage of causing negative aesthetics during use, in particular unpleasant taste and sensations and stain promotion. For example, chlorhexidine is one of the most effective antimicrobials, but local side effects notably unpleasant taste and staining, limit its acceptability and long term use. In addition chlorohexidine and similar antibiotic actives such as doxycycline and metronidazole may have potential bacterial resistance issues along with a more widespread organism killing potential, i.e., both harmful and beneficial bacteria. For this reason and because consumers generally prefer products based on natural or naturally occurring ingredients, there is an advantage in developing oral care products based on actives such as those derived from plant essential oils. Many of these essential oil actives are GRAS materials known to be safe for ingestion and effective to provide antimicrobial activity without harming beneficial oral microbial flora. However, many essential oil actives, particularly those with antimicrobial activity have strong, overpowering, or unsuitable flavor character, particularly when used at high levels to provide the desired activity. For example, thymol is well-known for its antimicrobial activity and has been utilized in oral care preparations in sufficient quantities to provide beneficial therapeutic effects. While thymol provides beneficial therapeutic effects, it also provides the consumer with a flavor perception that can be described as unpleasant, harsh or medicinal in taste. Other examples include citral and eugenol, both also having antimicrobial activity but having respectively, a strong lemon and strong spicy clove flavor, which may not be preferred for certain applications.

The present invention is thus directed at formulating compositions that utilize one or more derivatives of essential oil compounds to replace or reduce the amount of the parent essential oil compounds themselves, as the derivatives have been found to provide therapeutic effects without the drawback of the unacceptable, unpleasant or overpowering taste or odor associated with the parent or underivatized essential oil compound. In one aspect, the present invention provides oral, nasal and throat care products comprising a base flavor composition comprising one or more derivatives of plant essential oil compounds, such base flavor composition providing therapeutic benefits including antimicrobial activity. The base flavor has a mild taste and aroma and can be easily blended with other typical flavoring agents such as mint oils, fruit oils, menthol, and coolants to provide pleasant tasting products that encourage user compliance with prescribed use.

In another aspect, antimicrobial topical compositions for use on skin, hair and other mucosal surfaces are provided utilizing a perfume blend comprising selected essential oil derivatives.

### SUMMARY OF THE INVENTION

The present invention is directed to compositions comprising one or more derivatives of essential oil compounds for use in personal care compositions, such as compositions for oral, throat and skin care. These derivatives include acetals of essential oil aldehydes and ketones; esters or ethers of essential oil alcohols and phenolics; and esters of essential oil acids. Examples of parent essential oil aldehydes and ketones to derivatize include citral, cinnamic aldehyde, ρ-anisaldehyde, vanillin, ethyl vanillin, heliotropin, carvone, and menthone. Examples of parent essential oil alcohols and phenolics include thymol, eugenol, isoeugenol, dihydroeugenol, carvacrol, carveol, geraniol, nerol, vanillyl alcohol, heliotropyl alcohol, ρ-anisyl alcohol, cinnamyl alcohol and β-ionol. Examples of parent essential oil acids include ρ-anisic acid, cinnamic acid, vanillic acid and geranic acid. The present compositions comprising essential oil derivatives are useful as base flavor or base perfume for incorporation in personal care products and to provide other benefits including antimicrobial efficacy. Optionally the compositions will comprise additional antimicrobially-effective or anti-inflammatory components including those also obtained from plant essential oils or synthetic versions thereof.

Oral, nasal and throat care products include products in powder, paste or liquid forms, which on being used are retained for a time sufficient to contact the surfaces and the internal mucous membranes of the oral or nasal cavities or the pharynx. Examples of such products include mouthwashes, dental and throat lozenges, gargles, chewing gum, dentifrice or toothpastes, throat sprays, toothpicks, dental tablets and powders and topical solutions for application in dental treatment, as well as cough syrups, chewable antacids and digestion promoting preparations. The present compositions comprising essential oil derivatives may also be incorporated in compositions for topical application to the skin, hair and other mucosal surfaces including lotions or creams, skin cleansers, shampoos and conditioners, cosmetic products such as lipsticks and foundations, wipes and towelettes and feminine hygiene products such as menstrual pads and tampons.

These and other features, aspects, and advantages of the present invention will become evident to those skilled in the art from the detailed description which follows.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral care composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition may be in various forms including toothpaste, dentifrice, tooth gel, subgingival gel, mouthrinse, mousse, foam, denture product, mouthspray, lozenge, chewable tablet or chewing gum. The oral care composition may also be incorporated onto strips or films for direct application or attachment to oral surfaces.

The term “dentifrice”, as used herein, includes paste, gel, liquid, powder or tablet formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “teeth” refers to natural teeth as well as artificial teeth or dental prosthesis.

Herein, the terms “tartar” and “calculus” are used interchangeably and refer to mineralized dental plaque biofilms.

The term “nasal and throat care composition” or “respiratory compositions” refer to compositions for use to treat respiratory or throat conditions and which can be used herein in a form that is deliverable to a mammal in need. Non-limiting examples include liquid compositions, nasal compositions, beverage, supplemental water, pills, soft gels, tablets, capsules, gel compositions, foam compositions, and combinations thereof. Nasal compositions, liquid compositions, gel compositions can be in a form that is directly deliverable to the nose, mouth and throat. These compositions and/or preparations can be delivered by a delivery device selected from droppers, pump, sprayers, liquid dropper, cup, bottle, liquid filled gel, liquid filled gummy, center filled gum, chews, films, center filled lozenge, gum filled lozenge, pressurized sprayers, atomizers, air inhalation devices, liquid filled compressed tablet, liquid filled gelatin capsule, liquid filled capsule, and other packaging and equipment, and combinations thereof. The sprayer, atomizer, and air inhalation devices can be associated with a battery or electric power source. For example, the respiratory compositions can be used to provide long lasting, instant or on demand cough or sore throat relief to a human.

The term “instant” and/or “on demand” as used herein refers to the compositions providing relief of one or more symptoms that is being treated, prevented, alleviated, ameliorated, inhibited, or mitigated within 20 minutes of application, alternatively within 15 minutes of application, alternatively within 10 minutes of application, alternatively within 5 minutes of application, alternatively within 2 minutes of application, alternatively within 1 minute of application. Long-lasting as used herein refers to the compositions providing relief of one or more symptoms that is being treated, prevented, alleviated, ameliorated, inhibited, or mitigated for a period of up to about 24 hours, alternatively about 12 hours, alternatively about 6 hours, alternatively about 4 hours.

The terms “pharmaceutically-acceptable carrier” or “orally-acceptable carrier” refer to safe and effective materials and conventional additives used in personal care compositions. For example, materials used in oral care compositions include but are not limited to one or more of fluoride ion sources, anti-calculus or anti-tartar agents, buffers, abrasives such as silica, alkali metal bicarbonate salts, thickening materials, humectants, water, surfactants, titanium dioxide, flavor system, sweetening agents, xylitol, and coloring agents.

The term “essential oils” as used herein refers to oils or extracts distilled or expressed from plants and to constituents of these oils. Typical essential oils and their main constituents are those obtained for example from thyme (thymol, carvacrol), oregano (carvacrol, terpenes), lemon (limonene, terpinene, phellandrene, pinene, citral), lemongrass (citral, methylheptenone, citronellal, geraniol), orange flower (linalool, β-pinene, limonene), orange (limonene, citral), anise (anethole, safrol), clove (eugenol, eugenyl acetate, caryophyllene), rose (geraniol, citronellol), rosemary (borneol, bornyl esters, camphor), geranium (geraniol, citronellol, linalool), lavender (linalyl acetate, linalool), citronella (geraniol, citronellol, citronellal, camphene), eucalyptus (eucalyptol); peppermint (menthol, menthyl esters), spearmint (carvone, limonene, pinene); wintergreen (methyl salicylate), camphor (safrole, acetaldehyde, camphor), bay (eugenol, myrcene, chavicol), cinnamon (cinnamic aldehyde, cinnamyl acetate, eugenol), tea tree (terpinen-4-ol, cineole), and cedar leaf (α-thujone, β-thujone, fenchone). Essential oils are widely used in perfumery and as flavorings, medicine and solvents. Essential oils, their composition and production, are described in detail in Kirk-Othmer Encyclopedia of Chemical Technology, 4^{th} Edition and in The Merck Index, 13^{th} Edition.

Active and other ingredients useful herein may be categorized or described by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

The essential and optional components of the present compositions are described in the following paragraphs.

In one embodiment of the present invention, oral care compositions are provided comprising one or more derivatives of essential oil aldehydes and ketones including citral, neral, geranial, cinnamic aldehyde, ρ-anisaldehyde, vanillin, ethyl vanillin, heliotropin, carvone, and menthone; essential oil alcohols and phenolics including thymol, eugenol, isoeugenol, dihydroeugenol, carvacrol, carveol, geraniol, nerol, vanillyl alcohol, heliotropyl alcohol, ρ-anisyl alcohol, cinnamyl alcohol and β-ionol; and essential oil acids including ρ-anisic acid, cinnamic acid, and vanillic acid. Examples of derivatives herein include ester, ether, or acetal derivatives of the above essential oil compounds. These derivatives are easily prepared using standard synthesis methods starting from essential oil extracts containing the essential oil compounds as main constituents, such as oils of lemongrass, citrus (orange, lemon, lime), citronella, geranium, rose, eucalyptus, oregano, bay and clove; isolated or purified constituents of these essential oils or synthetic versions thereof. For example, synthesis of ester and ether derivatives of essential oil alcohols or phenolics such as carvacrol and eugenol is described in Lett. Appl. Microbiol., 43, 149-154 (2006) and J. Chem. Crystallogr. 39:655-661 (2009).

Use of the derivatives of essential oil compounds is advantageous in that the derivatives generally possess less flavor and/or odor impact compared to their respective parent compounds, thereby providing milder flavors and aromas that can be tailored more easily for each desired application. For example, eugenol derivatives instead of eugenol may be used in the present compositions. Eugenol is the principal phenolic component of the essential oil extracted from cloves (Syzygium aromaticum) and is useful because of its many pharmacological activities including antiseptic, analgesic, antimicrobial, antifungal and anti-inflammatory. However, eugenol has an extremely strong spicy flavor and aroma and typically must be used sparingly such as in oral care compositions. Ester and ether derivatives of eugenol such as eugenyl acetate, eugenyl formate, eugenyl benzoate, eugenol methyl ether, and eugenol amyl ether by comparison have milder flavor and odor, and would permit use at higher levels without contributing undesirable taste or aroma characteristics. For example, sensory testing among an expert sensory panel and consumers demonstrated a taste preference for products with eugenyl acetate vs. those with eugenol. Eugenyl acetate was also perceived in sensory testing as having significantly less of a eugenol or clove-like taste and odor compared to eugenol. This sensory testing is conducted by a Descriptive Profile Panel (DPP) comprised of 10 individuals who have been screened for above average sensory acuity and ability to describe sensations such as taste and smell. The panelists are trained on oral care product specific attributes via Spectrum™ methodology and have the unique ability to evaluate oral care products on an attribute by attribute basis without regard to personal preference or bias. In this test, panelists swished and expectorated with the test and control solutions (50 ppm eugenol or 50 ppm eugenyl acetate in 5% sucrose water solution, filtered water as control) and rated the perception of sweet (contributed by sucrose) and eugenol (clove-like) taste at key time points (immediately after expectoration, 5 minutes after expectoration, and 15 minutes after expectoration). Panelists also provided descriptive analysis and additional voluntary comments to call out any other sensory attributes. Each attribute is measured on a 0-60 scale with half-unit intervals. Using this scale, the expert panel rated the 50 ppm eugenol solution 24.5 and the 50 ppm eugenyl acetate solution 14.2, i.e., a difference of 10.3. In this test, absolute attribute rating differences of 7.5 or more represent a likely consumer-meaningful difference for that attribute. Therefore, the perception of eugenol or clove-like taste in the eugenyl acetate solution was significantly and meaningfully lower than that of the eugenol solution.

Increased chemical stability is another advantage of using certain derivatives of essential oil compounds. Many of the desirable essential oil compounds are chemically unstable in the presence of light, air, moisture, high temperatures and oxidizing environments. For example, the chemical instability of citral is in part due to the aldehyde group, which is susceptible to chemical reaction and degradation. One way to derivatize citral involves protecting the aldehyde group. A useful derivative of citral may be prepared by transforming the aldehyde group to an acetal group, yielding citral acetals, such as citral dimethyl acetal and citral diethyl acetal. Citral acetals may be prepared by reacting citral with an excess of an alcohol (ROH) in the presence of acid. In use in an aqueous environment, the acetal groups in the citral acetal compounds hydrolyze yielding the parent citral compound, which provides the antimicrobial activity.

Typically, flavor and perfume ingredients are plant-sourced oils and extracts, constituents isolated therefrom or synthetic versions thereof. Many of these essential oil extracts or individual chemical components have been reported to have antimicrobial activity. However, the activity of individual components is typically too weak to be of practical use, unless combined with other antimicrobials or used at fairly high concentrations. However at high concentrations, these flavor chemicals may introduce flavor notes and aroma that may be incompatible with the overall flavor perception desired in the final product. Thus, the use of derivatives that have milder or less flavor or odor impact provides formulation flexibility in that the amount of each component in the composition can be adjusted to derive maximum consumer appeal in terms of flavor, taste and aroma while providing other benefits such as antimicrobial efficacy. For example, it has been found that flavor compositions comprising one or more derivatives of the essential oil compounds listed above, provide effective antimicrobial activity as well as an acceptable and pleasant taste when incorporated into oral and throat care products such as dentifrice, mouthrinse and throat spray. Taste is of course an important attribute for oral and throat care products, since these require fairly long residence time in the mouth for efficacy. For example, some compositions may comprise one or a mixture of an acetal derivative of citral such as citral diethyl acetal, citral dimethyl acetal or citral propylene glycol acetal or an ester or ether derivative of eugenol or isoeugenol such as eugenyl acetate, isoeugenyl acetate, eugenol ethyl ether or isoeugenol ethyl ether.

Another example of derivatives useful herein are thymol derivatives as they contribute antimicrobial activity without necessarily the organoleptic negatives associated with thymol itself that can be described as unpleasant, harsh or medicinal in taste. Examples of thymol derivatives useful herein include thymyl acetate and thymol methyl ether. Use of thymol derivatives would eliminate the need to mask the taste of thymol to improve consumer acceptability of the product. Masking the unpleasant taste of thymol in oral hygiene compositions is disclosed for example, in U.S. Pat. No. 4,945,087 to Talwar, et al. by using a sugar alcohol or a mixture with anethole.

One embodiment of the present invention provides base flavor compositions for incorporation in oral care products, comprising a blend of essential oil compounds and derivatives thereof, the blend comprising a first component selected from acyclic or non-ring structures such as citral, geraniol, nerol or derivatives thereof and a second component selected from ring-containing structures such as eugenol, isoeugenol, dihydroeugenol, carvacrol, thymol, carvone, cinnamic aldehyde, ρ-anisaldehyde, vanillin, ethyl vanillin, heliotropin or derivatives thereof. Essential oils may be used to provide the above essential oil compounds or derivatives including oils of lemongrass, citrus (orange, lemon, lime), citronella, geranium, rose, eucalyptus, oregano, bay and clove. Or the essential oil compounds may be provided as individual or purified chemicals rather than supplied in the composition by addition of natural oils or extracts as these sources may contain other components that may be unstable with other components of the composition or may introduce flavor notes that are incompatible with the desired flavor profile resulting in a less acceptable product from an organoleptic standpoint. Natural oils or extracts that have been purified or concentrated to contain mainly the desired component(s) are particularly useful herein.

The blend will comprise 2, 3, 4, 5 or more of the above essential oil compounds including, one or more derivatives thereof. Greater synergy in terms of antimicrobial efficacy may be obtained the more different components are blended together particularly when the blend comprises at least one non-ring structure (e.g., citral or a citral derivative) and at least one ring structure (e.g., eugenol or a derivative such as eugenyl acetate). A blend may comprise at least two ring structures or at least two non-ring structures. Still another may comprise three ring structures or three non-ring structures. For example, a blend comprising two non-ring structures (citral or a derivative and geraniol or a derivative) and eugenyl acetate as the ring structure is found to have high efficacy against oral bacteria. Another blend may comprise two non-ring structures (e.g., citral or a derivative and geraniol or a derivative) and three ring structures (e.g., eugenyl acetate, eucalyptol and carvacrol or a carvacrol derivative).

Particularly useful herein are essential oil compounds and their derivatives that have been demonstrated to have activity against microorganisms known to be involved in undesirable oral cavity conditions such as gingivitis, periodontal disease and oral malodor, in particular bacteria such as P. gingivalis and F. nucleatum and other oral cavity strains including B. forsythus, A. actinomycetemcomitans, T. denticola, T. socranskii, P. intermedia, L. acidophilus, L. casei, A. viscosus, S. sobrinus, S sanguis, S. viridans, and S. mutans.

Periodontal disease may involve one or more of the following conditions: inflammation of the gingiva, formation of periodontal pockets, bleeding and/or pus discharge from the periodontal pockets, resorption of alveolar bone, loose teeth and loss of teeth. Bacteria present in dental plaque which forms on the surface of the teeth and in the periodontal pocket contribute to both the initiation and progress of periodontal disease. Thus, in order to prevent or treat periodontal disease, these bacteria must be suppressed by some means other than simple mechanical scrubbing. Towards this end, there has been a great deal of research aimed at developing therapeutic dentifrices, mouthwashes, and methods of treating periodontal disease, which are effective in suppressing these bacteria. However, periodontal disease involves more than just the bacterial infection. Severe periodontal disease involves the destruction of periodontal tissue, which is primarily caused by the indirect effects mediated by the host's reaction to the bacteria in the periodontium and gingival sulcus, specifically inflammation of the gingival and periodontium, or gingivitis. If left unchecked, gingivitis may progress into periodontitis, which may result in attachment loss, bone destruction and tooth loss. Anaerobic bacteria are generally regarded as the initiating agent of gingivitis, with subsequent progression and disease severity determined by the host immune response, i.e., inflammation, which is a nonspecific cellular and biochemical process involving multiple pro-inflammatory agents.

Bacterial metabolites induce leukocyte chemotaxis which results in the accumulation of inflammatory cells at the site of the bacterial challenge. Furthermore, bacterial metabolites induce the production of inflammatory mediators by leukocytic cells, in particular monocytes. Amongst these are local disease mediators such as metabolites of arachidonic acid, e.g., leukotrienes, prostaglandins and thromboxanes. Prostaglandins have been found to be particularly involved in the metabolism and destruction of tissue and alveolar bone. Indeed, the production of prostaglandins in the periodontal tissues has been found to be a key mediator of the loss of alveolar bone in the periodontium. Patients with periodontal breakdown show an elevated prostaglandin E₂ (PGE₂) level both in the gingival tissue as well as in the crevicular fluid. Prostaglandins and thromboxanes are formed from arachidonic acid by an enzyme cascade, the first step of which is the cyclooxygenation by an enzyme called cyclooxygenase (COX). Inhibiting the cyclooxygenase would inhibit the formation of prostaglandins and thus reduce alveolar bone loss. Indeed certain cyclooxygenase inhibitors, particularly non steroidal anti-inflammatory drugs such as indomethacin and flurbiprofen have been found to markedly reduce the resorption of alveolar bone. Once inflammation starts, the process can self-propagate even when the causative agents, i.e., bacteria are removed. Therefore, an effective therapy for gingivitis would desirably include the combination of an antibacterial agent and an anti-inflammatory agent. Such combinations are disclosed for example in commonly assigned U.S. patent application Ser. No. 11/595,530, published as US 2007/0053849A1. The actives disclosed therein include those having both antibacterial and anti-inflammatory activities.

Thus, essential oil compounds exhibiting both antibacterial and anti-inflammatory activities, including citral, geraniol, cinnamic aldehyde, ρ-anisaldehyde, eugenol, dihydroeugenol, eucalyptol, carvacrol, and thymol are useful herein, particularly in the form of their derivatives. The activities of the underivatized or parent essential oil compounds have been demonstrated using the assays described in the above cited publication US 2007/0053849A1 and in U.S. application Ser. No. 12/062,870, published as US2008/0253976A1, including inhibitory activity against one or more of bacterial virulence and/or inflammation factors involving bacteria such as P. gingivalis; inhibition of biofilm growth; and germ-kill efficacy.

The present base flavor composition comprising one or more derivatives of essential oil compounds is used at levels of at least about 0.02%, typically from about 0.05% to about 5.00% in finished oral care products. In some embodiments, the base flavor is present at levels of from about 0.05% to about 2.0%, from about 0.1% to about 1.5%, from about 0.3% to about 1.0%, or from about 0.5% to about 0.8% by weight of the oral care composition.

To provide antimicrobial activity, the base flavor composition will typically comprise one or more derivative compounds blended with other essential oil compounds, including those listed above having both antimicrobial and anti-inflammatory activities. The antimicrobial blend will comprise at least about 0.5% by weight of each component, at least about 1%, at least about 5%, or at least about 10% in some embodiments. In two component blends, the weight ratio of the first component, i.e., a derivative compound, to the second component, i.e., a second derivative compound or a parent essential oil compound may range from 5:95 to 95:5. For example, a two component blend may contain a geraniol derivative (e.g., geranyl acetate, geranyl propionate or geraniol butyl ether) and eucalyptol at a 65:35 ratio. Another blend may contain a eugenol derivative (e.g., eugenyl acetate, eugenyl benzoate or eugenol methyl ether) and citral at a 50:50 ratio. Another blend may contain a eugenol derivative and a citral derivative (e.g., citral dimethyl acetal or citral diethyl acetal) at a ratio ranging from 2:1 to 1:2. A three component blend may contain e.g., a eugenol derivative, citral and eucalyptol. A four component blend may add geraniol or derivative to the previous three component blend. A five component blend may add thymyl acetate to the four component blend above. A six component blend may comprise for example, from about 1.5% to about 20% citral; from about 10% to about 50% geraniol; from about 10% to about 40% eucalyptol; from about 2% to about 25% eugenyl acetate; from about 2% to about 10% thymyl acetate and from about 2% to about 20% carvacrol or derivative (e.g., carvacryl acetate, carvacrol ethyl ether or carvacrol methyl ether). Table 1 below lists nonlimiting examples of essential oil compounds and their derivatives useful herein.

TABLE 1
Parent Essential Oil Compounds and Their Derivatives

| Parent Essential Oil Compound | Examples of Derivative(s) |
|---|---|
| Thymol [5-methyl-2-(1-methylethyl)phenol] | Thymol methyl ether; thymyl esters (acetate; |
| | isobutyrate; 2-methylbutyrate; 1 isovalerate) |
| Carvacrol [2-methyl-5-(1-methylethyl)phenol] | Carvacrol ethyl ether; carvacrol methyl ether; |
| | carvacryl acetate |
| Eugenol [2-methoxy-4-(2-propenyl)phenol] | Eugenol ethers (n-amyl ether; methyl; isoamyl); |
| | eugenyl esters (acetate; formate; benzoate; |
| | phenylacetate) |
| Isoeugenol [2-methoxy-4-(prop-1-enyl)phenol | Isoeugenol methyl ether; isoeugenol ethyl ether; |
| | isoeugenyl acetate |
| Dihydroeugenol [2-methoxy-4-propylphenol] | Dihydroeugenyl acetate |
| Cinnamyl alcohol [3-phenylprop-2-enol] | Cinnamyl esters (acetate; formate; propionate; |
| | valerate; butyrate; acetoacetate) |
| Cinnamic acid [3-phenylprop-2-enoic acid] | Amyl cinnamate; benzyl cinamate; butyl |
| | cinnamate; ethyl cinnamate; methyl cinnamate; |
| | propyl cinnamate |
| Cinnamic aldehyde [3-phenylprop-2-enal] | Cinnamic aldehyde acetals (diethyl acetal; dimethyl |
| | acetal; propylene glycol acetal) |
| ρ-Anisyl alcohol [4-methoxybenzyl alcohol] | ρ-anisyl esters (acetate; acetoacetate; butyratel |
| | isobutyrate; propionate; valerate; isovalerate) |
| ρ-Anisic acid [4-methoxybenzoic acid] | Ethyl ρ-anisate; methyl ρ-anisate; |
| ρ-Anisaldehyde [4-methoxybenzaldehyde] | ρ-anisaldehyde acetals (propylene glycol acetal; |
| | dimethyl acetal; diethyl acetal; 2,3-butanediol |
| | acetal |
| Vanillin [4-hydroxy-3-methoxybenzaldehyde] | Vanillin propylene glycol acetal; vanillyl acetate; |
| | vanillyl isobutyrate; vanillin ethyl ether; vanilln |
| | butyl ether |
| Vanillic acid [4-hydroxy-3-methoxybenzoic acid] | Ethyl vanillate |
| Ethyl vanillin [3-ethoxy-4-hydroxybenzaldehyde] | Ethyl vanillin propylene glycol acetal; ethyl |
| | vanillin hexylene glycol acetal; ethyl vanillyl |
| | isobutyrate |
| Heliotropin [3,4-dihydroxybenzaldehyde | Heliotropin propylene glycol acetal |
| methylene ketal] | |
| Heliotropyl alcohol [1,3-benzodioxol-5- | Heliotropyl esters (acetate, propionate, |
| ylmethanol] | isobutyrate ) |
| Carveol [2-methyl-5-prop-1-en-2-ylcyclohex-2- | Carvyl esters (acetate; propionate; butyrate; |
| en-1-ol] | formate; isovalerate) |
| β-Ionol [4-(2,6,6-trimethyl-1-cyclohexenyl)but-3- | β-Ionyl acetate |
| en-2-ol] | |
| Carvone [2-methyl-5-propen-2-ylcyclohex-2- | Carvone acetals (dimethyl, acetal; diethyl acetal; |
| enone | propylene glycol acetal; butylene glycol acetal) |
| Citral [3,7-dimethyl-2,6-octadienal) 2:1 mixture | Citral dimethyl acetal; citral diethyl acetal; citral |
| of geranial and neral | glyceryl acetal; citral propylene glycol acetal |
| Geraniol [3,7-dimethyl-2,6-octadien-1-ol] | Geraniol butyl ether; geranyl esters (acetate; |
| | acetoacetate; isovalerate; propionate) |
| Geranic acid [(2Z)-3,7-dimethylocta-2,6-dienoic | Ethyl geranate; methyl geranate |
| acid] | |
| Nerol[(2Z)-3,7-dimethylocta-2,6-dien-1-ol] | Nerol ethyl ether; nerol methyl ether; neryl esters |
| | (acetate; formate; butyrate; isobutyrate; hexanoate; |
| | isovalerate; propionate) |

A series of studies were conducted to evaluate the antimicrobial efficacy of compositions comprising the present essential oil derivatives using in vivo test methods described below. Test products for these studies were made by formulating eugenyl acetate/citral, eugenol/citral acetal or eugenol/citral blend(s) in a fluoride toothpaste base also containing zinc and stannous salts (such as shown in Examples IIj to IIo below). The control product was Crest™ Anticavity fluoride toothpaste sold by Procter & Gamble. Results of testing compositions containing blends of essential oil compounds demonstrated their antimicrobial efficacy as summarized in Table 2 below. All test compositions were statistically better than the control treatment which did not contain the subject essential oil compound(s) and derivative(s) thereof, but were not statistically different from one another. The data demonstrate that the derivatives of the essential oil compounds provide equivalent antimicrobial efficacy as their parent compounds.

Volatile sulfur compounds (VSC's) in morning breath of subjects are evaluated by a Halimeter after use of test product(s). Germ kill is evaluated by standard microbiological assays.

One study is a randomized, single blind crossover study using a panel of approximately 12 subjects. Subjects are randomized across test products for each 24-hour treatment period. Each period lasts from baseline (morning Day 0) to next day (morning Day 1). Subjects brush with their assigned treatments unsupervised three times on Day 0 (after morning baseline measurements, late afternoon and at bedtime) of each period. Subjects are asked to refrain from eating or drinking for 30 minutes following treatment. Subjects are not allowed to brush with another toothbrush or paste, use mouthwash, floss teeth or chew gum during the treatment phase of the study.

Breath odor (Halimeter) and germ kill (tongue) assessments are made at baseline (morning Day 0) and final at 24 hrs (morning of Day 1). All measurements are taken in the morning. Subjects are instructed to abstain from eating, drinking, or performing any oral hygiene from bedtime the night before measurement until after breath measurements are made. Subjects are assessed for volatile sulfur compound emissions (VSC's) utilizing a commercially available portable instrument called a Halimeter (Interscan Corporation, CA). This instrument is sensitive to hydrogen sulfide and methyl mercaptan, two of the primary components of foul breath odor. Results are reported in ppb of these VSC's.

For bacteria kill assessments, a supragingival plaque sample is taken from each subject by moving a sterile synthetic cotton swab along the gingival gum line of the upper buccal surfaces. The swab is transferred to a vial containing 10 mL of a microbiological transport fluid (Dey/Engley neutralizing broth or DE broth). The sample is appropriately diluted (1:100, 1:1,000 and 1:10,000 final) with sterile DE broth and spiral plated onto ETSA-NV selective agar plates and ETSA plates to enumerate total Gram-negative anaerobes (GNA) and total facultative anaerobes (TFA) present on/at the gingival margin. Results are reported as log colony forming units (log cfu) per ml.

TABLE 2
Antimicrobial Efficacy of Compositions

| | | | 2:1 | 2:1 | 1:1 | |
|---|---|---|---|---|---|---|
| | | 1:1 | Eugenyl Acetate: | Eugenyl | Eugenol: | |
| in vivo Test | | Eugenol: | Citral Diethyl | Acetate: | Citral Dimethyl | Control |
| Method | Units | Citral | Acetal | Citral | Acetal | Treatment |
| Volatile Sulfur | ppb | 211 | 198 | 190 | — | 294 |
| Compounds (VSC) | ppb | 142 | — | — | 122 | 203 |
| Germ Kill (TFA) | log CFU/ml | 7.2 | — | — | 7.02 | 7.56 |
| Germ Kill (GNA) | log CFU/ml | 5.84 | — | — | 5.71 | 6.34 |

Another study to evaluate antimicrobial efficacy used the in vivo Plaque Growth Regrowth Model (PGRM) described in D. J. White, et al. “A New Plaque Glycolysis and Regrowth Method (PGRM) for the In Vitro Determination of Antimicrobial Dentifrice/Rinse Efficacy towards the Inhibition of Plaque Growth and Metabolism—Method Development, Validation, and Initial Activity Screens”. J. Clin Dent. Vol VI, Sp. Issue: 59-70, 1995. This method uses approximately 12 subjects for testing of experimental products and measurement of bacterial metabolism and activity. Subjects brush lingually and then swish their dentition with test dentifrice to expose overnight dental plaque to the dentifrice. Plaque glycolysis and regrowth of plaque samples taken with a sterile cotton swab immediately before, 15 minutes after and 45 minutes after treatment with dentifrice are measured by the method described by White et al. Swab samples are appropriately prepped and analyzed for plaque glycolysis and plaque regrowth. Plaque glycolysis is a measure of bacterial viability, i.e., plaque bacteria produce organic acids such as lactic acid, acetic acid and butyric acid. Plaque regrowth is measured via optical density (OD) of the sample indicating turbidity or presence of bacterial mass. Less bacterial regrowth is indicated by a lower OD or turbidity of the sample. Results of testing the above compositions containing blends of essential oil compounds using the PGRM method demonstrated their antimicrobial efficacy as indicated by less acid production (i.e., less bacterial activity) and lower turbidity (less bacterial growth) compared to a control treatment.

Additional antimicrobially-effective and/or anti-inflammatory components, may optionally be included in the present compositions. Such other antimicrobially-effective and/or anti-inflammatory components may include one or more of flavor/fragrance chemicals such as o-cymen-5-ol (isopropylmethylphenol, IPMP), farnesol, benzyl alcohol, benzaldehyde, hinokitiol (isopropyltropolone), terpinene-4-ol, zingerone, allyl isothiocyanate, cuminaldehyde, dipentene, α-pinene, β-pinene, menthol, methyl salicylate, anethole, limonene, ocimene, n-decyl alcohol, citronellal, citronellol, menthyl acetate, citronellyl acetate, linalool, ethyl linalool, camphor, safrole, chlorothymol, guaiacol, phenol, phenyl salicylate , guaiacol, 5-propenylguaethol, 4-ethyl-2-methoxyphenol, 4-allyl-2-methoxyphenol acetate, and 4-methyl guaiacol. Additional useful components having anti-inflammatory activity include flavonoids and flavones such as baicalein, baicalin, wogonoside, wogonin, and quercetin; phenolics such as catechin, gallocatechin gallate, epicatechin (EC), epigallocatechin (EGC), epigallocatechin gallate (EGCG), epicatechin gallate (ECG), theaflavine, thearubigins, anthocyanidins/proanthocyanidins and anthocyanins (e.g., cyanidin, delphinidin, pelargonidin, peonidin, malvidin and petunidin); tannic acid; gallic acid; ellagic acid; ellagitannins; hexamidine; and berberine. Natural sources of these chemicals may be used including oils, extracts or essences of spearmint, peppermint, wintergreen, lemon, orange, lime, cherry, sage, rosemary, cinnamon, cassia, oregano, ginger, basil, coriander, cilantro, allspice rose, tea tree (Melaleuca), pimento, laurel, anise, fennel, cumin, bay, bergamot, bitter almond, citronella, coal tar, lavender, mustard fennel, pine, pine needle, cedar leaf, sassafras, cubeb, spike lavender, creosote, horseradish, wasabi, tea, cranberry, pomegranate, oak bark and the like.

The present compositions may optionally include additional ingredients such as mint-type oils (spearmint, peppermint, wintergreen), fruit oils, spice oils, coolants and sweeteners as part of the flavor system.

Suitable cooling agents or coolants for use herein include a wide variety of materials such as menthol and derivatives thereof. Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds such as N-ethyl-ρ-menthan-3-carboxamide, known commercially as “WS-3”, and others in the series such as WS-5 (N-ethoxycarbonylmethyl-ρ-menthan-3-carboxamide), WS-12 [N-(4-methoxyphenyl)-ρ-menthan-3-carboxamide] and WS-14 (N-tert-butyl-ρ-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”. Additional suitable coolants include 3-1-menthoxypropane-1,2-diol known as TK-10, isopulegol (under the tradename Coolact P) and ρ-menthane-3,8-diol (under the tradename Coolact 38D) all available from Takasago; menthone glycerol acetal known as MGA; menthyl esthers such as menthyl acetate, menthyl acetoacetate, menthyl lactate known as Frescolat® supplied by Haarmann and Reimer, and monomenthyl succinate under the tradename Physcool from V. Mane. The terms menthol and menthyl as used herein include dextro- and levorotatory isomers of these compounds and racemic mixtures thereof TK-10 is described in U.S. Pat. No. 4,459,425, Amano et al. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide. Other N-substituted ρ-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N-((5-methyl-2-(1-methylethyl)cyclohexyl)carbonyl)alanine ethyl ester. Menthyl esters including those of amino acids such as glycine and alanine are disclosed e.g., in EP 310,299 and in U.S. Pat. Nos. 3,111,127; 3,917,613; 3,991,178; 5,5703,123; 5,725,865; 5,843,466; 6,365,215; 6,451,844; and 6,884,903. Ketal derivatives are described, e.g., in U.S. Pat. Nos. 5,266,592; 5,977,166 and 5,451,404. Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H. R. Watson, et al. J. Soc. Cosmet. Chem. (1978), 29, 185-200 and R. Eccles, J. Pharm. Pharmacol., (1994), 46, 618-630.

Suitable sweeteners include those well known in the art, including both natural and artificial sweeteners. Some suitable water-soluble sweeteners include monosaccharides, disaccharides, polysaccharides and derivatives such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, glycyrrhizin, xylitol and erythritol. Suitable water-soluble artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include Dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as thaumatoccous danielli (Thaumatin I and II) can be used. A composition may contain from about 0.1% to about 10% of sweetener, or from about 0.1% to about 1%, by weight of the composition.

The flavor system may also include salivating agents, warming agents, and numbing agents. These agents are present in the compositions at a level of from about 0.001% to about 10%, or from about 0.1% to about 1%, by weight of the composition. Suitable salivating agents include Jambu® manufactured by Takasago and Optaflow® from Symrise. Examples of warming agents are capsicum and nicotinate esters, such as benzyl nicotinate. Suitable numbing agents include benzocaine, lidocaine, clove bud oil, and ethanol.

In addition to the components described above, the present compositions may comprise additional optional components collectively referred to as orally acceptable carrier materials, which are described in the following paragraphs.

### Orally Acceptable Carrier Materials

Orally acceptable carrier materials include one or more compatible solid or liquid excipients or diluents which are suitable for topical oral administration. By “compatible” is meant that the components of the composition are capable of being commingled without interaction in a manner which would substantially reduce composition stability and/or efficacy.

The carriers or excipients of the present invention can include the usual and conventional components of dentifrices, non-abrasive gels, subgingival gels, mouthwashes or rinses, mouth sprays, chewing gums, lozenges and breath mints as more fully described hereinafter.

The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, etc. as disclosed in e.g., U.S. Pat. No. 3,988,433 to Benedict. Carrier materials for biphasic dentifrice formulations are disclosed in U.S. Pat. Nos. 5,213,790; 5,145,666 and 5,281,410 all to Lukacovic et al. and in U.S. Pat. Nos. 4,849,213 and 4,528,180 to Schaeffer. Mouthwash, rinse or mouth spray carrier materials typically include water, flavoring and sweetening agents, etc., as disclosed in, e.g., U.S. Pat. No. 3,988,433 to Benedict. Lozenge carrier materials typically include a candy base; chewing gum carrier materials include a gum base, flavoring and sweetening agents, as in, e.g., U.S. Pat. No. 4,083,955 to Grabenstetter et al. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g. U.S. Pat. Nos. 5,198,220 and 5,242,910 both to Damani. Carriers suitable for the preparation of compositions of the present invention are well known in the art. Their selection will depend on secondary considerations like taste, cost, and shelf stability, etc.

The compositions of the present invention may also be in the form of non-abrasive gels and subgingival gels, which may be aqueous or non-aqueous. In still another aspect, the invention provides a dental implement impregnated with the present composition. The dental implement comprises an implement for contact with teeth and other tissues in the oral cavity, said implement being impregnated with the present composition. The dental implement can be impregnated fibers including dental floss or tape, chips, strips, films and polymer fibers.

In one embodiment, the compositions of the subject invention are in the form of dentifrices, such as toothpastes, tooth gels, tooth powders and tablets. Components of such toothpaste and tooth gels generally include one or more of a dental abrasive (from about 6% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion) and an anticalculus agent (from about 0.1% to about 13%). Tooth powders, of course, contain substantially all non-liquid components.

Other embodiments of the subject invention are liquid products, including mouthwashes or rinses, mouth sprays, dental solutions and irrigation fluids. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%). Components of dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

Types of orally acceptable carriers or excipients which may be included in compositions of the present invention, along with specific non-limiting examples, are discussed in the following paragraphs.

### Other Active Agents

The present compositions may optionally include other agents, such as other antimicrobial agents. Included among such agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, and triclosan monophosphate. The quaternary ammonium agents include those in which one or two of the substituents on the quaternary nitrogen has a carbon chain length (typically alkyl group) from about 8 to about 20, typically from about 10 to about 18 carbon atoms while the remaining substitutents (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexa hydropyrimidine, benzalkonium chloride, benzethonium chloride and methyl benzethonium chloride are exemplary of typical quaternary ammonium antibacterial agents. Other compounds are bis[4-(R-amino)-1-pyridinium]alkanes as disclosed in U.S. Pat. No. 4,206,215, issued to Bailey. Other antimicrobials such as copper salts, zinc salts and stannous salts may also be included. Also useful are enzymes, including endoglycosidase, papain, dextranase, mutanase, and mixtures thereof. Such agents are disclosed in U.S. Pat. No. 2,946,725 to Norris et al. and in U.S. Pat. No. 4,051,234 to Gieske et al. Preferred antimicrobial agents include zinc salts, stannous salts, cetyl pyridinium chloride, chlorhexidine, triclosan, triclosan monophosphate, and flavor oils. Triclosan and other agents of this type are disclosed in Parran, Jr. et al., U.S. Pat. No. 5,015,466 and U.S. Pat. No. 4,894,220 to Nabi et al. These agents provide anti-plaque benefits and are typically present at levels of from about 0.01% to about 5.0%, by weight of the composition.

Another optional active agent that may be added to the present compositions is a dentinal desensitizing agent to control hypersensitivity, such as salts of potassium, calcium, strontium and tin including nitrate, chloride, fluoride, phosphates, pyrophosphate, polyphosphate, citrate, oxalate and sulfate.

### Anticalculus Agent

The present compositions may optionally include an anticalculus agent, such as a pyrophosphate salt as a source of pyrophosphate ion. The pyrophosphate salts useful in the present compositions include the dialkali metal pyrophosphate salts, tetraalkali metal pyrophosphate salts, and mixtures thereof. Examples include disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, alternatively less than about 10% of the total pyrophosphate dissolved in the composition. For example, tetrasodium pyrophosphate salt is used in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, generally from about 1.5% to about 15%, from about 2% to about 10%, or from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, Third Edition, Volume 17, Wiley-Interscience Publishers (1982).

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., as well as, e.g., polyamino propane sulfonic acid (AMPS), diphosphonates (e.g., EHDP; AHP), polypeptides (such as polyaspartic and polyglutamic acids), and mixtures thereof.

### Fluoride Source

It is common to have a fluoride compound present in dentifrices and other oral compositions in an amount sufficient to give a fluoride ion concentration in the composition, and/or when it is used of from about 0.0025% to about 5.0% by weight, alternatively from about 0.05% to about 2.0% by weight, to provide anticaries effectiveness. A wide variety of fluoride ion-yielding materials can be employed as sources of soluble fluoride in the present compositions. Examples of suitable fluoride ion-yielding materials are found in U.S. Pat. No. 3,535,421 to Briner et al. and U.S. Pat. No. 3,678,154 to Widder et al. Representative fluoride ion sources include: stannous fluoride, sodium fluoride, potassium fluoride, sodium monofluorophosphate, indium fluoride, amine fluoride and many others.

### Abrasives

Dental abrasives useful in the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are particularly useful because of their unique benefits of exceptional dental cleaning and polishing performance without unduly abrading tooth enamel or dentine. The silica abrasive polishing materials herein, as well as other abrasives, may have an average particle size ranging between about 0.1 to about 30 microns, and typically from about 3 to about 20 microns. The abrasive can be precipitated silica or silica gels such as the silica xerogels described in Pader et al., U.S. Pat. No. 3,538,230 and DiGiulio, U.S. Pat. No. 3,862,307. Examples include the silica xerogels marketed under the trade name “Syloid” by the W.R. Grace & Company, Davison Chemical Division and precipitated silica materials such as those marketed by the J. M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are described in more detail in Wason, U.S. Pat. No. 4,340,583; and in commonly-assigned U.S. Pat. Nos. 5,603,920; 5,589,160; 5,658,553; 5,651,958; and 6,740,311.

Mixtures of abrasives can be used such as mixtures of the various grades of Zeodent® silica abrasives listed above. The total amount of abrasive in dentifrice compositions of the subject invention may range from about 6% to about 70% by weight; toothpastes typically contain from about 10% to about 50% of abrasives. Dental solution, mouth spray, mouthwash and non-abrasive gel compositions of the subject invention typically contain little or no abrasive.

### Tooth Substantive Agent

The present invention may include a tooth substantive agent such as polymeric surface active agents (PMSA's), which are polyelectrolytes, more specifically anionic polymers. The PMSA's contain anionic groups, e.g., phosphate, phosphonate, carboxy, or mixtures thereof, and thus, have the capability to interact with cationic or positively charged entities. The “mineral” descriptor is intended to convey that the surface activity or substantivity of the polymer is toward mineral surfaces such as calcium phosphate minerals or teeth.

PMSA's are useful in the present compositions because of their stain prevention benefit. It is believed the PMSA's provide a stain prevention benefit because of their reactivity or substantivity to mineral surfaces, resulting in desorption of portions of undesirable adsorbed pellicle proteins, in particular those associated with binding color bodies that stain teeth, calculus development and attraction of undesirable microbial species. The retention of these PMSA's on teeth can also prevent stains from accruing due to disruption of binding sites of color bodies on tooth surfaces.

The ability of PMSA's to bind stain promoting ingredients of oral care products, for example, stannous ions and cationic antimicrobials, is also believed to be helpful. The PMSA will also provide tooth surface conditioning effects which produce desirable effects on surface thermodynamic properties and surface film properties, which impart improved clean feel aesthetics both during and most importantly, following rinsing or brushing. Many of these polymeric agents are also known or expected to provide tartar control benefits when applied in oral compositions, hence providing improvement in both the appearance of teeth and their tactile impression to consumers.

Desired surface effects include: 1) creating a hydrophilic tooth surface immediately after treatment; and 2) maintaining surface conditioning effects and control of pellicle film for extended periods following product use, including post brushing or rinsing and throughout more extended periods. The effect of creating an increased hydrophilic surface can be measured in terms of a relative decrease in water contact angles. The hydrophilic surface, importantly, is maintained on the tooth surface for an extended period after using the product.

The polymeric mineral surface active agents include any agent which will have a strong affinity for the tooth surface, deposit a polymer layer or coating on the tooth surface and produce the desired surface modification effects. Suitable examples of such polymers are polyelectrolytes such as condensed phosphorylated polymers; polyphosphonates; copolymers of phosphate- or phosphonate-containing monomers or polymers with other monomers such as ethylenically unsaturated monomers and amino acids or with other polymers such as proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); polycarboxylates and carboxy-substituted polymers; and mixtures thereof. Suitable polymeric mineral surface active agents include the carboxy-substituted alcohol polymers described in U.S. Pat. Nos. 5,292,501; 5,213,789, 5,093,170; 5,009,882; and 4,939,284; all to Degenhardt et al. and the diphosphonate-derivatized polymers in U.S. Pat. No. 5,011,913 to Benedict et al; the synthetic anionic polymers including polyacrylates and copolymers of maleic anhydride or acid and methyl vinyl ether (e.g., Gantrez), as described, for example, in U.S. Pat. No. 4,627,977, to Gaffar et al. Examples include diphosphonate modified polyacrylic acid. Polymers with activity have sufficient surface binding propensity to desorb pellicle proteins and remain affixed to enamel surfaces. For tooth surfaces, polymers with end or side chain phosphate or phosphonate functions are useful although other polymers with mineral binding activity may prove effective depending upon adsorption affinity.

Additional examples of suitable phosphonate containing polymeric mineral surface active agents include the geminal diphosphonate polymers disclosed as anticalculus agents in U.S. Pat. No. 4,877,603 to Degenhardt et al; phosphonate group containing copolymers disclosed in U.S. Pat. No. 4,749,758 to Dursch et al. and in GB 1,290,724 (both assigned to Hoechst) suitable for use in detergent and cleaning compositions; and the copolymers and cotelomers disclosed as useful for applications including scale and corrosion inhibition, coatings, cements and ion-exchange resins in U.S. Pat. No. 5,980,776 to Zakikhani et al. and U.S. Pat. No. 6,071,434 to Davis et al. Additional polymers include the water-soluble copolymers of vinylphosphonic acid and acrylic acid and salts thereof disclosed in GB 1,290,724 wherein the copolymers contain from about 10% to about 90% by weight vinylphosphonic acid and from about 90% to about 10% by weight acrylic acid, more particularly wherein the copolymers have a weight ratio of vinylphosphonic acid to acrylic acid of 70% vinylphosphonic acid to 30% acrylic acid; 50% vinylphosphonic acid to 50% acrylic acid; or 30% vinylphosphonic acid to 70% acrylic acid. Other suitable polymers include the water soluble polymers disclosed by Zakikhani and Davis prepared by copolymerizing diphosphonate or polyphosphonate monomers having one or more unsaturated C═C bonds (e.g., vinylidene-1,1-diphosphonic acid and 2-(hydroxyphosphinyl)ethylidene-1,1-diphosphonic acid), with at least one further compound having unsaturated C═C bonds (e.g., acrylate and methacrylate monomers). Suitable polymers include the diphosphonate/acrylate polymers supplied by Rhodia under the designation ITC 1087 (Average MW 3000-60,000) and Polymer 1154 (Average MW 6000-55,000).

Useful PMSA's will be stable with other components of the oral care composition such as ionic fluoride and metal ions. Also useful are polymers that have limited hydrolysis in high water content formulations, thus permitting a simple single phase dentifrice or mouthrinse formulation. If the PMSA does not have these stability properties, one option is a dual phase formulation with the polymeric mineral surface active agent separated from the fluoride or other incompatible component. Another option is to formulate non-aqueous, essentially non-aqueous or limited water compositions to minimize reaction between the PMSA and other components.

Among useful PMSA's herein are polyphosphates. A polyphosphate is generally understood to consist of two or more phosphate molecules arranged primarily in a linear configuration, although some cyclic derivatives may be present. Although pyrophosphates (n=2) are technically polyphosphates, particularly useful polyphosphates are those having around three or more phosphate groups so that surface adsorption at effective concentrations produces sufficient non-bound phosphate functions, which enhance the anionic surface charge as well as hydrophilic character of the surfaces. Examples of inorganic polyphosphate salts include tripolyphosphate, tetrapolyphosphate and hexametaphosphate, among others. Polyphosphates larger than tetrapolyphosphate usually occur as amorphous glassy materials. The linear polyphosphates are represented by the formula:

XO(XPO₃)ₙX

wherein X is sodium, potassium or ammonium and n averages from about 3 to about 125. Some commercially available polyphosphates are those having n averaging from about 6 to about 21, such as those known as Sodaphos (n≈6), Hexaphos (n≈13), and Glass H (n≈21) and manufactured by FMC Corporation and Astaris. These polyphosphates may be used alone or in combination. Polyphosphates are susceptible to hydrolysis in high water formulations at acid pH, particularly below pH 5. Thus longer-chain polyphosphates are useful, in particular Glass H with an average chain length of about 21. It is believed such longer-chain polyphosphates when undergoing hydrolysis produce shorter-chain polyphosphates which are still effective to deposit onto teeth and provide a stain preventive benefit.

Other polyphosphorylated compounds may be used in addition to or instead of the polyphosphate, in particular polyphosphorylated inositol compounds such as phytic acid [myo-inositol 1,2,3,4,5,6-hexakis (dihydrogen phosphate)], myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof. Herein, the term “phytate” includes phytic acid and its salts as well as the other polyphosphorylated inositol compounds.

The amount of tooth substantive agent will typically be from about 0.1% to about 35% by weight of the total oral composition. In dentifrice formulations, the amount typically ranges from about 2% to about 30%, or from about 5% to about 25%, or from about 6% to about 20%. In mouthrinse compositions, the amount of tooth substantive agent may range from about 0.1% to 5% or from about 0.5% to about 3%.

In addition to creating the surface modifying effects, the tooth substantive agent may also function to solubilize insoluble salts. For example, Glass H has been found to solubilize insoluble stannous salts. Thus, in compositions containing stannous fluoride for example, Glass H contributes to decreasing the stain promoting effect of stannous.

### Chelating Agents

Another optional agent is a chelating agent, also called sequestrants, such as gluconic acid, tartaric acid, citric acid and pharmaceutically-acceptable salts thereof. Chelating agents are able to complex calcium found in the cell walls of the bacteria. Chelating agents can also disrupt plaque by removing calcium from the calcium bridges which help hold this biomass intact. However, it is not desired to use a chelating agent which has an affinity for calcium that is too high, as this may result in tooth demineralization, which is contrary to the objects and intentions of the present invention. Suitable chelating agents will generally have a calcium binding constant of about 10¹ to 10⁵ to provide improved cleaning with reduced plaque and calculus formation. Chelating agents also have the ability to complex with metallic ions and thus aid in preventing their adverse effects on the stability or appearance of products. Chelation of ions, such as iron or copper, helps retard oxidative deterioration of finished products.

Examples of suitable chelating agents are sodium or potassium gluconate and citrate; citric acid/alkali metal citrate combination; disodium tartrate; dipotassium tartrate; sodium potassium tartrate; sodium hydrogen tartrate; potassium hydrogen tartrate; sodium, potassium or ammonium polyphosphates and mixtures thereof. The chelating agent may be used from about 0.1% to about 2.5%, from about 0.5% to about 2.5% or from about 1.0% to about 2.5% in certain embodiments.

Still other chelating agents suitable for use in the present invention are the anionic polymeric polycarboxylates. Such materials are well known in the art, being employed in the form of their free acids or partially or fully neutralized water soluble alkali metal (e.g. potassium and sodium) or ammonium salts. Examples are 1:4 to 4:1 copolymers of maleic anhydride or acid with another polymerizable ethylenically unsaturated monomer, such as methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates are disclosed in U.S. Pat. No. 4,138,477 to Gaffar and U.S. Pat. No. 4,183,914 to Gaffar et al. and include copolymers of maleic anhydride with styrene, isobutylene or ethyl vinyl ether; polyacrylic, polyitaconic and polymaleic acids; and sulfoacrylic oligomers of M.W. as low as 1,000 available as Uniroyal ND-2.

### Surfactants

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458. The present composition typically comprises an anionic surfactant at a level of from about 0.025% to about 9%, from about 0.05% to about 5% or from about 0.1% to about 1%.

Another suitable surfactant is one selected from the group consisting of sarcosinate surfactants, isethionate surfactants and taurate surfactants. Examples for use herein include alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the present compositions from about 0.1% to about 2.5% or from about 0.5% to about 2.0% by weight.

Cationic surfactants useful in the present invention include derivatives of quaternary ammonium compounds having one long alkyl chain containing from about 8 to 18 carbon atoms such as lauryl trimethylammonium chloride; cetyl pyridinium chloride; cetyl trimethylammonium bromide; coconut alkyltrimethylammonium nitrite; cetyl pyridinium fluoride; etc. Quaternary ammonium fluorides having detergent properties are described in U.S. Pat. No. 3,535,421 to Briner et al. Certain cationic surfactants can also act as germicides in the compositions disclosed herein. Cationic surfactants such as chlorhexidine, although suitable for use in the current invention, may not be preferred due to their capacity to stain the oral cavity's hard tissues.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al. Typical alkyl dimethyl betaines include decyl betaine or 2-(N-decyl-N,N-dimethylammonio) acetate, coco betaine or 2-(N-coco-N,N-dimethyl ammonio) acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, and lauramidopropyl betaine.

### Thickening Agents

In preparing toothpaste or gels, thickening agents are added to provide a desirable consistency to the composition, to provide desirable active release characteristics upon use, to provide shelf stability, and to provide stability of the composition, etc. Suitable thickening agents include one or a combination of carboxyvinyl polymers, carrageenan, hydroxyethyl cellulose (HEC), natural and synthetic clays (e.g., Veegum and laponite) and water soluble salts of cellulose ethers such as sodium carboxymethylcellulose (CMC) and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

Suitable carboxyvinyl polymers useful as thickening or gelling agents include carbomers which are homopolymers of acrylic acid crosslinked with an alkyl ether of pentaerythritol or an alkyl ether of sucrose. Carbomers are commercially available from B.F. Goodrich as the Carbopol® series, including Carbopol 934, 940, 941, 956, and mixtures thereof.

Thickening agents are typically present in an amount from about 0.1% to about 15%, from about 2% to about 10%, or from about 4% to about 8%, by weight of the total toothpaste or gel composition, can be used. Higher concentrations may be used for chewing gums, lozenges and breath mints, sachets, non-abrasive gels and subgingival gels.

### Humectants

Another optional carrier material of the present compositions is a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, may comprise from about 0% to about 70% or from about 5% to about 25%, by weight of the compositions herein. Suitable humectants for use in compositions of the subject invention include edible polyhydric alcohols such as glycerin, sorbitol, xylitol, butylene glycol, polyethylene glycol, propylene glycol and trimethyl glycine.

### Miscellaneous Carrier Materials

Water employed in the preparation of commercially suitable oral compositions would desirably be of low ion content and free of organic impurities. Water may comprise up to about 99% by weight of the aqueous compositions herein. These amounts of water include the free water which is added plus that which is introduced with other materials, such as with sorbitol.

The present invention may also include an alkali metal bicarbonate salt, which may serve a number of functions including abrasive, deodorant, buffering and adjusting pH. Alkali metal bicarbonate salts are soluble in water and unless stabilized, tend to release carbon dioxide in an aqueous system. Sodium bicarbonate, also known as baking soda, is a commonly used alkali metal bicarbonate salt. The present composition may contain from about 0.5% to about 30% by weight of an alkali metal bicarbonate salt.

The pH of the present compositions may be adjusted through the use of buffering agents. Buffering agents, as used herein, refer to agents that can be used to adjust the pH of aqueous compositions such as mouthrinses and dental solutions typically to a range of about pH 4.0 to about pH 8.0. Buffering agents include sodium bicarbonate, monosodium phosphate, trisodium phosphate, sodium hydroxide, sodium carbonate, sodium acid pyrophosphate, citric acid, and sodium citrate and are typically included at a level of from about 0.5% to about 10% by weight.

Poloxamers may be employed in the present compositions. A poloxamer is classified as a nonionic surfactant and may also function as an emulsifying agent, binder, stabilizer, and other related functions. Poloxamers are difunctional block-polymers terminating in primary hydroxyl groups with molecular weights ranging from 1,000 to above 15,000. Poloxamers are sold under the tradename of Pluronics and Pluraflo by BASF including Poloxamer 407 and Pluraflo L4370.

Other emulsifying agents that may be used include polymeric emulsifiers such as the Pemulen® series available from B.F. Goodrich, and which are predominantly high molecular weight polyacrylic acid polymers useful as emulsifiers for hydrophobic substances.

Titanium dioxide may also be added to the present compositions as coloring or opacifying agent typically at a level of from about 0.25% to about 5% by weight.

Other optional agents that may be used in the present compositions include dimethicone copolyols selected from alkyl- and alkoxy-dimethicone copolyols, such as C₁₂ to C₂₀ alkyl dimethicone copolyols and mixtures thereof, as aid in providing positive tooth feel benefits. One example is cetyl dimethicone copolyol marketed under the trade name Abil EM90. The dimethicone copolyol is generally present from about 0.01% to about 25%, from about 0.1% to about 5%, or from about 0.5% to about 1.5% by weight.

### Respiratory Ingredients

The personal care compositions for nasal and throat care can comprise a wide range of respiratory ingredients. Nonlimiting examples include analgesics, anticholinergics, antihistamines, anti-inflammatories, antipyretics, antitussives, antivirals, decongestants, expectorants, mucolytics, and combinations thereof.

Example of decongestants include: oxymetazoline, phenylephrine, xylometazoline, naphazoline, 1-desoxyephedrine, ephedrine, propylhexedrine, pseudoephedrine, and phenylpropanolamine Example of anticholinergics include: ipratropium, chlorpheniramine, brompheniramine, diphenhydramine, doxylamine, clemastine, and triprolidine. Common analgesics, anti-inflammatories and antipyretics include: ibuprofen, ketoprofen, diclofenac, naproxen, acetaminophen, and aspirin. Example of antivirals include: amantidine, rimantidine, pleconaril, zanamivir, and oseltamivir. Examples of antitussives include codeine, dextromethorphan, chlophedianol and levodropropizine. Examples of expectorants include guaifenesin. Examples of mucolytics include ambroxol and N-acetylcysteine. Examples of antihistamines include diphenhydramine, doxylamine, triprolidine, clemastine, pheniramine, chlorpheniramine, brompheniramine, Dexbrompheniramine, loratadine, cetirizine and fexofenadine, Amlexanox, Alkylamine Derivatives, Cromolyn, Acrivastine, Ibudilast, Bamipine, Ketotifen, Nedocromil, Omalizumab, Dimethindene, Oxatomide, Pemirolast, Pyrrobutamine, Pentigetide, Thenaldine, Picumast, Tolpropamine, Ramatroban, Triprolidine, Repirinast, Suplatast Tosylate Aminoalkylethers, Tazanolast, Bromodiphenhydramine, Tranilast, Carbinoxamine, Traxanox, Chlorphenoxamine, Diphenhydramine, Diphenylpyaline, Doxylamine, Embramine, p-Methyldiphenhydramine, Moxastine, Orphenadrine, Phenyltoloxamine, Setastine, Ethylenediamine Derivatives, Chloropyramine, Chlorothen, Methapyrilene, Pyrilamine, Talastine, Thenyldiamine, Thonzylamine Hydrochloride, Tripelennamine, piperazines, Chlorcyclizine, Clocinizine, Homochlorcyclizine, Hydroxyzine, Tricyclics, Phenothiazines, Mequitazine, Promethazine, Thiazinamium Methylsulfate, Other Tricyclics, Azatadine, Cyproheptadine, Deptropine, Desloratadine, Isothipendyl, Olopatadine, Rupatadine, Antazoline, Astemizole, Azelastine, Bepotastine, Clemizole, Ebastine, Emedastine, Epinastine, Levocabastine, Mebhydroline, Mizolastine, Phenindamine, Terfenadine, Tritoqualine.

The composition may comprise an amount of respiratory ingredient in the range of from about 0% to about 15%, alternatively 0.0001% to about 10%, alternatively from about 0.001% to about 7%, and alternatively from about 0.01% to about 5%, all by weight of the composition.

### Method of Use

The present invention also relates to methods for controlling bacterial activity in the oral cavity, which cause undesirable conditions including plaque, caries, calculus, gingivitis, periodontal disease and malodor. The benefits of these compositions may increase over time when the composition is used repeatedly.

The method of use or treatment herein may comprise contacting a subject's dental enamel and mucosal surfaces with the oral compositions according to the present invention. The method may comprise brushing with a dentifrice or rinsing with a dentifrice slurry or mouthrinse. Other methods include contacting a topical oral gel, denture product, mouthspray, or other form with the subject's teeth and oral mucosa. The subject may be any person or animal in need of treatment or prevention of the above undesirable conditions and whose tooth surfaces are contacted with the oral composition. By animal is meant to include household pets or other domestic animals, or animals kept in captivity.

For example, a method of treatment may include a person brushing a dog's teeth with one of the dentifrice compositions. Another example would include rinsing a cat's mouth with an oral composition for a sufficient amount of time to see a benefit. Pet care products such as chews and toys may be formulated to contain the present oral compositions. The composition may be incorporated into a relatively supple but strong and durable material such as rawhide, ropes made from natural or synthetic fibers, and polymeric articles made from nylon, polyester or thermoplastic polyurethane. As the animal chews, licks or gnaws the product, the incorporated active elements are released into the animal's oral cavity into a salivary medium, comparable to an effective brushing or rinsing.

Other methods of use include cleansing and disinfecting hands and skin using sanitizing compositions or wipes containing the present antimicrobial blend of essential oil materials. Or a throat spray containing the present blend may be used to treat a throat infection or sore throat.

When the composition is a respiratory composition the term “orally administering” and/or “administering” with respect to the human/mammal means that the human/mammal ingests or is directed to ingest, or does ingest, or deliver, or chew, or drink, or spray, or place in mouth, one or more of the present respiratory composition. The human/mammal may be directed to deliver the respiratory composition to the site that the human/mammal intends to treat for example the mouth and/or throat. The human/mammal may be directed to ingest or deliver or chew, or drink, or spray, or place in mouth the composition, such direction and or deliver may be that which instructs and/or informs the human that use of the composition may and/or will provide relief from the respiratory symptom (e.g., symptomatic relief, whether temporary or permanent) for example, relief from coughing and/or sore throat. The relief can be long-lasting, instant or on demand. For example, such direction may be oral direction (e.g., through oral instruction from, for example, a physician, pharmacist, or other health professional), radio or television media (e.g., advertisement), or written direction (e.g., through written direction from, for example, a physician, pharmacist, or other health professional (e.g., scripts), sales professional organization (e.g., through, for example, marketing brochures, pamphlets, or other instructive paraphernalia), written media (e.g., internet, electronic mail, or other computer-related media)), and/or packaging associated with the composition (e.g., a label present on a delivery device holding the preparation). As used herein, “written” means through words, pictures, symbols, and/or other visible or tactile descriptors. Such information need not utilize the actual words used herein, for example, “respiratory”, “symptom”, or “mammal”, but rather use of words, pictures, symbols, tactile means, and the like conveying the same or similar meaning are contemplated within the scope of this invention.

In a further embodiment, the respiratory composition is directed to methods of treating and providing sore throat or cough relief on demand comprising administering a preparation as described herein to a mammal in need of such treatment. As further used herein, “treatment” and/or “providing relief”, with respect to cough relief or sore throat, mean that administration of the referenced respiratory preparation prevents, alleviates, ameliorates, inhibits, or mitigates one or more symptoms of the condition, such as sore throat.

The present invention can also be directed to methods of “prevention” including preventing a cough or its associated symptoms from occurring in a mammal, for example when the mammal is predisposed to acquiring the symptoms of coughing, inhibiting the onset of coughing or its associated symptoms; and/or alleviating, reversing, or curing the coughing episode or its associated symptoms.

Administration may be on an as-needed or as-desired basis, for example, once-monthly, once-weekly, or daily, including multiple times daily, for example, at least once daily, from one to about six times daily, from about two to about four times daily, or about three times daily. The amount of respiratory composition administered may be dependent on a variety of factors, including the general quality of health of the mammal, age, gender, weight, or severity of symptoms.

### EXAMPLES

The following examples further describe and demonstrate embodiments within the scope of the present invention. These examples are given solely for the purpose of illustration and are not to be construed as limitations of the present invention as many variations thereof are possible without departing from the spirit and scope.

### Example I

### Essential Oil Blends

Compositions Ia to Ik shown below are prepared by blending parent essential oil compounds and one or more derivatives with other essential oils or extracts. These essential oil blends may be used as base flavor or base perfume in personal care compositions such as those exemplified herein.

| Ingredient | Ia | Ib | Ic | Id | Ie | If | Ig | Ih | Ii | Ij | Ik |
|---|---|---|---|---|---|---|---|---|---|---|---|
| Peppermint Oil | | | | | | | 55 | 59 | | | 10 |
| Spearmint Oil | 50 | | | | 62 | 55 | | | 77 | | |
| Wintergreen | | 50 | 52 | | | | | | | 9 | |
| Oil | | | | | | | | | | | |
| Cinnamon Oil | | | | 63 | | | | | | | |
| Carvacrol | | | | | | | | | | 1 | 10 |
| Citral | 16.5 | | | | | 6 | | | | | |
| Citral Di- | | 25 | | | | 6.3 | | | | 30 | |
| methyl Acetal | | | | | | | | | | | |
| Citral Diethyl | | | | | | | | | 8 | | |
| Acetal | | | | | | | | | | | |
| Eugenol | | 25 | | | | | | | | | |
| Eugenyl | 33.5 | | | | | | 21 | | 15 | 60 | 40 |
| Acetate | | | | | | | | | | | |
| Methyl | | | | | 19 | | | | | | |
| Eugenol | | | | | | | | | | | |
| Methyl | | | | 19 | | | | 17 | | | |
| Isoeugenol | | | | | | | | | | | |
| ρ-Anisalde- | | | | | 6 | | | | | | 40 |
| hyde | | | | | | | | | | | |
| Anisyl | | | 10 | | | | | 12 | | | |
| Butyrate | | | | | | | | | | | |
| ρ-Anisalde- | | | | | 13 | | | | | | |
| hyde Propylene | | | | | | | | | | | |
| Glycol Acetal | | | | | | | | | | | |
| Cinnamyl | | | 16 | | | | | 12 | | | |
| Butyrate | | | | | | | | | | | |
| Cinnamic Al- | | | | 18 | | | | | | | |
| dehyde Di- | | | | | | | | | | | |
| methyl Acetal | | | | | | | | | | | |
| Vanillyl | | | | | | 16 | | | | | |
| Isobutyrate | | | | | | | | | | | |
| Vanillyl | | | 10 | | | | | | | | |
| Acetate | | | | | | | | | | | |
| Heliotropyl | | | 12 | | | 16.7 | | | | | |
| Acetate | | | | | | | | | | | |
| Heliotropyl | | | | | | | 24 | | | | |
| Propionate | | | | | | | | | | | |

### Example II

### Dentifrice Compositions

Dentifrice compositions according to the present invention IIa-IIo are shown below with amounts of ingredients in weight %. These compositions are made using conventional methods. In consumer sensory tests, oral care compositions according to the present invention were rated as having a pleasant, long-lasting, natural, light herbal taste and providing cleaning and freshening of the mouth without burn and unpleasant aftertaste.

| Ingredient | IIa | IIb | IIc | IId | IIe | IIf | IIIg | Ih | IIi |
|---|---|---|---|---|---|---|---|---|---|
| Peppermint Oil | | | | | | | 1.0 | 1.0 | |
| Spearmint Oil | 1.0 | | | | 1.0 | 1.0 | | | 1.0 |
| Wintergreen Oil | | 1.0 | 1.0 | | | | | | |
| Cinnamon Oil | | | | 1.0 | | | | | |
| Citral | 0.33 | | | | | 0.1 | | | |
| Citral Dimethyl Acetal | | 0.5 | | | | 0.1 | | | |
| Citral Diethyl Acetal | | | | | | | | | 0.1 |
| Eugenol | | 0.5 | | | | | | | |
| Eugenyl Acetate | 0.67 | | | | | | 0.2 | | 0.2 |
| Methyl Eugenol | | | | | 0.3 | | | | |
| Methyl Isoeugenol | | | | 0.1 | | | | 0.3 | |
| Anisyl Butyrate | | | 0.2 | | | | | 0.2 | |
| ρ-Anisaldehyde | | | | | 0.2 | | | | |
| Propylene Glycol Acetal | | | | | | | | | |
| Cinnamyl Butyrate | | | 0.3 | | | | | 0.2 | |
| Cinnamic Aldehyde | | | | 0.3 | | | | | |
| Dimethyl Acetal | | | | | | | | | |
| Vanillyl Isobutyrate | | | | | | 0.3 | | | |
| Vanillyl Acetate | | | 0.2 | | | | | | |
| Heliotropyl Acetate | | | | | | 0.3 | | | |
| Heliotropyl Propionate | | | | | | | 0.4 | | |
| Ethyl Vanillyl Acetate | | | | | 0.1 | | | | |
| Ethyl Vanillyl | | | 0.2 | | | | | | |
| Isobutyrate | | | | | | | | | |
| Eucalyptol | | | | 0.1 | | | 0.2 | | |
| Geraniol | | | | 0.1 | | | | | |
| Sodium Fluoride | 0.243 | 0.243 | 0.243 | 0.243 | 0.243 | 0.243 | 0.243 | 0.243 | 0.243 |
| Sorbitol 70% Solution | 65.0 | 65.0 | 65.0 | 65.0 | 65.0 | 65.0 | 65.0 | 65.0 | 65.0 |
| Sodium Lauryl Sulfate | 4.0 | 4.0 | 4.0 | 4.0 | 4.0 | 4.0 | 4.0 | 4.0 | 4.0 |
| 28% Soln | | | | | | | | | |
| Na Saccharin | 0.4 | 0.4 | 0.4 | 0.4 | 0.4 | 0.4 | 0.4 | 0.4 | 0.4 |
| Silica Abrasive | 20.0 | 20.0 | 20.0 | 20.0 | 20.0 | 20.0 | 20.0 | 20.0 | 20.0 |
| NaOH 50% Soln | 0.7 | 0.7 | 0.7 | 0.7 | 0.7 | 0.7 | 0.7 | 0.7 | 0.7 |
| Sodium Acid | 1.3 | 1.3 | 1.3 | 1.3 | 1.3 | 1.3 | 1.3 | 1.3 | 1.3 |
| Pyrophosphate | | | | | | | | | |
| Xanthan Gum | 0.2 | 0.2 | 0.2 | 0.2 | 0.2 | 0.2 | 0.2 | 0.2 | 0.2 |
| Carbomer 956 | 0.4 | 0.4 | 0.4 | 0.4 | 0.4 | 0.4 | 0.4 | 0.4 | 0.4 |
| Sodium CMC | 0.2 | 0.2 | 0.2 | 0.2 | 0.2 | 0.2 | 0.2 | 0.2 | 0.2 |
| Water | Q.S. | Q.S. | Q.S. | Q.S. | Q.S. | Q.S. | Q.S. | Q.S. | Q.S. |

| Ingredient | IIj | IIk | IIl | IIm | IIn | IIo |
|---|---|---|---|---|---|---|
| Peppermint Oil | | 0.5 | 0.7 | | 0.3 | 0.3 |
| Spearmint Oil | | 0.5 | 0.3 | 0.7 | 0.7 | 0.7 |
| Wintergreen Oil | 0.7 | | | | | |
| Cinnamon Oil | 0.3 | | | 0.3 | 0.3 | |
| Citral | | | | | 0.33 | |
| Citral Dimethyl Acetal | 0.25 | | | | | |
| Citral Diethyl Acetal | | | | | | 0.33 |
| Eugenol | | | 0.1 | | | |
| Eugenyl Acetate | 0.5 | | | | 0.67 | 0.67 |
| Methyl Eugenol | | | 0.2 | | | |
| Methyl Isoeugenol | | | | 0.3 | | |
| Anisyl Butyrate | | | | 0.2 | | |
| ρ-Anisaldehyde Propylene Glycol Acetal | | 0.2 | 0.2 | | | |
| Cinnamyl Butyrate | | 0.3 | | | | |
| Cinnamic Aldehyde Dimethyl Acetal | | | | 0.2 | | |
| Heliotropyl Acetate | | 0.2 | | | | |
| Heliotropyl Propionate | | | | | | |
| Ethyl Vanillyl Acetate | | | 0.1 | | | |
| Ethyl Vanillyl Isobutyrate | | 0.2 | | | | |
| Eucalyptol | | | | 0.05 | | |
| Geraniol | | | | 0.05 | | |
| Zinc Citrate Dihydrate | 0.788 | 0.788 | 0.788 | 0.533 | 0.533 | 0.533 |
| Sodium Citrate Tribasic Dihydrate | 0.274 | 0.274 | 0.274 | | | |
| Sodium Gluconate | | | | 1.06 | 1.06 | 1.06 |
| Sodium Fluoride | 0.243 | 0.243 | 0.243 | 0.243 | 0.243 | 0.243 |
| Stannous Chloride | 0.21 | 0.21 | 0.21 | 1.16 | 1.16 | 1.16 |
| Sorbitol 70% Soln. | 40.5 | 40.5 | 40.5 | 38.0 | 38.0 | 38.0 |
| Sodium Saccharin | 0.3 | 0.3 | 0.3 | 0.5 | 0.5 | 0.5 |
| Hydroxyethyl cellulose | 0.3 | 0.3 | 0.3 | 0.5 | 0.5 | 0.5 |
| Sodium CMC | 1.3 | 1.3 | 1.3 | 1.3 | 1.3 | 1.3 |
| Carrageenan Mixture | 0.7 | 0.7 | 0.7 | 0.7 | 0.7 | 0.7 |
| Titanium Dioxide | 0.525 | 0.525 | 0.525 | 0.525 | 0.525 | 0.525 |
| Silica Abrasive | 17.0 | 17.0 | 17.0 | 14.5 | 14.5 | 14.5 |
| Sodium Alkyl Sulfate 28% soln. | 5.0 | 5.0 | 5.0 | 5.0 | 5.0 | 5.0 |
| Sodium Hydroxide 50% soln. | | | | 1.15 | 1.15 | 1.15 |
| Phytic Acid 50% soln. | | | | 0.8 | 0.8 | 0.8 |
| Color | | | | 0.3 | 0.3 | 0.3 |
| Water | Q.S | Q.S | Q.S | Q.S | Q.S | Q.S |

### Example III

### Mouthrinse Compositions

Mouthrinse compositions according to the present invention (IIIa-IIIj) are shown below with amounts of ingredients in weight %. These compositions are made using conventional methods. In consumer sensory tests, oral care compositions according to the present invention were rated as having a pleasant, long-lasting, natural, light herbal taste and providing cleaning and freshening of the mouth without burn and unpleasant aftertaste.

| Ingredient | IIIa | IIIb | IIIc | IIId | IIIe |
|---|---|---|---|---|---|
| Peppermint Oil | 0.1 | | | | |
| Citrus Oil | | 0.1 | | | |
| Wintergreen Oil | | | 0.1 | | |
| Cinnamon Oil | | | | 0.1 | |
| Citral | 0.033 | | | | |
| Citral Dimethyl Acetal | | 0.1 | | | |
| Eugenol | | 0.1 | | | |
| Eugenyl Acetate | 0.067 | | | | |
| Methyl Eugenol | | | | | 0.03 |
| Methyl Isoeugenol | | | | 0.02 | |
| Anisyl Butyrate | | | 0.04 | | |
| ρ-Anisaldehyde Propylene | | | | | 0.02 |
| Glycol Acetal | | | | | |
| Cinnamyl Butyrate | | | 0.06 | | |
| Cinnamic Aldehyde Dimethyl | | | | 0.05 | |
| Acetal | | | | | |
| Vanillyl Acetate | | | 0.06 | | |
| Ethyl Vanillyl Acetate | | | 0.04 | | 0.01 |
| Eucalyptol | | | | 0.01 | |
| Geraniol | | | | 0.01 | |
| Glycerin | 20.0 | 20.0 | 20.0 | 20.0 | 20.0 |
| Ethanol | 5.0 | 10.0 | | 20.0 | 20.0 |
| Poloxamer 407 | 1.0 | 0.5 | 1.0 | 0.5 | 0.5 |
| Na Saccharin | 0.05 | 0.03 | 0.03 | 0.05 | 0.05 |
| Cetylpyridinium Chloride | | 0.07 | | 0.07 | 0.07 |
| Water | Q.S. | Q.S. | Q.S. | Q.S. | Q.S. |

| Ingredient | IIIf | IIIg | IIIh | IIIi | IIIj |
|---|---|---|---|---|---|
| Peppermint Oil | | | 0.07 | 0.07 | 0.5 |
| Citrus Oil | | | | 0.03 | 0.5 |
| Wintergreen Oil | 0.1 | 0.07 | | | |
| Cinnamon Oil | | 0.05 | 0.03 | | |
| Citral | 0.01 | | | | 0.05 |
| Citral Dimethyl Acetal | 0.015 | | | | |
| Citral Diethyl Acetal | 0.01 | | | | |
| Eugenol | | | | 0.0025 | |
| Eugenyl Acetate | | 0.1 | | 0.02 | |
| Eugenol Methyl Ether | | | 0.03 | | 0.15 |
| Anisyl Butyrate | | | 0.02 | | |
| Cinnamyl Butyrate | | | 0.02 | | |
| Vanillyl Isobutyrate | 0.06 | | | | |
| Heliotropyl Acetate | 0.06 | | | | |
| Heliotropyl Propionate | | 0.05 | | | |
| Eucalyptol | | 0.03 | | | |
| Glycerin | 20.0 | 20.0 | 20.0 | 20.0 | 20.0 |
| Zinc Salt (Chloride, | 0.2 | 0.2 | 0.1 | 0.1 | 0.2 |
| Citrate or Lactate) | | | | | |
| Ethanol | 5.0 | 10.0 | | 20.0 | 20.0 |
| Poloxamer 407 | 1.0 | 0.5 | 1.0 | 0.5 | 0.5 |
| Na Saccharin | 0.05 | 0.03 | 0.03 | 0.05 | 0.05 |
| Cetylpyridinium Chloride | | 0.07 | | 0.07 | 0.07 |
| Water | Q.S. | Q.S. | Q.S. | Q.S. | Q.S. |

Additional examples of mouthrinse compositions (IIIk-IIIo) are shown below. These formulations are translucent, milky or cloudy emulsions rather than typically clear micellar solutions of oils. In these formulations, the flavor oils are not solubilized. Rather, they are stabilized small oil droplets dispersed in an aqueous external phase. The mean particle size of the oil droplets in these emulsions is typically in the range of about 100 nm to 1 μm in diameter, but may be outside this range depending on homogenization conditions. Advantages of formulating compositions as emulsions include: 1) the ability to load higher levels of oils without having to use more solvents, surfactants, or solubilizing agents; 2) the ability to use more hydrophobic flavors such as peppermint and spearmint (i.e., less water soluble relative to for example, wintergreen or cinnamon); 3) providing unique aesthetic qualities in terms of appearance and different mouth feel effects vs. typical solutions; and 4) importantly the ability to maintain high bioavailability of antimicrobials such as Cetyl Pyridinium Chloride (CPC) in the presence of high levels of hydropobic oils. The present mouthrinse emulsions have been demonstrated to maintain CPC bioavailability at about 80% or higher and antibacterial performance.

Bioavailability of the CPC in the formulations was evaluated using an in vitro Disk Retention Assay (DRA) as described in commonly assigned application WO 05/072693 and in S. J. Hunter-Rinderle, et al., “Evaluation of Cetylpyridinium Chloride-Containing Mouthwashes Using In Vitro Disk Retention and Ex Vivo Plaque Glycolysis Methods,” J. Clin. Den., 1997, 8:107-113. These assays are recommended for use in the proposed OTC monograph (Federal Register Vol. 68, No. 103 Part 356, “Oral Health Care Drug Products For Over-The-Counter Human Use; Antigingivitis/Antiplaque Drug Products; Establishment of a Monograph: Proposed Rules”). This method is designed as a performance assay to analyze mouthrinse formulations containing from about 0.03% to about 0.1% CPC to quantitatively determine the “free” (“unbound”) or “bioavailable” level of CPC needed for clinical efficacy. The DRA assay measures the amount of CPC “binding” to standardized cellulose filter disks during filtration of an undiluted mouthrinse sample. The “bioavailable” CPC binds to the hydroxyl groups on the cellulose fiber during filtration while the CPC which has been rendered “non-bioavailable” (or “bound”)” through interactions with mouthrinse components, simply passes through the filter paper, i.e., the positive charge on the compound is no longer available for binding to the negatively charged cellulose disks. In this way, the DRA test provides an estimate of the amount of CPC available for binding to bacteria and mucosal surfaces during use of the mouthrinse. DRA measurements of CPC availability have been positively correlated to the results of in vitro microbiological assays and in vivo germ kill tests. Historically, cellulose fibers have been used in other applications to similarly monitor biological activity of drug actives (“Dairy Products” in Official Methods of Analysis of the Association of Chemical Analytical Chemists. 13^{th} ed., 1980, Chapter 16:256). “Bioavailable” CPC is the amount of CPC bound to or adsorbed to cellulose disks. This is determined by measuring the differences in CPC concentration in the mouthrinse before and after exposure to standardized cellulose disks. The method has been validated and shown to perform with acceptable accuracy, precision, and selectivity.

The mouthrinse emulsions may be prepared as follows:

### Emulsion Concentrate:

1) Dissolve flavor oils and Vitamin E acetate in a small portion of ethanol.
2) Dissolve Cetyl Pyridinium Chloride (CPC) in a small portion of water.
3) Slowly add mixture of step 2) to mixture of step 1) with stirring.
4) Homogenize the mixture of step 3) using a processor such as a MicroFluidizer® available from MicroFluidics, Newton, Mass.

### Final Mix

5) Combine remaining water, ethanol, glycerin and sweetener.
6) Combine concentrate of step 4) to mixture of step 5 to create the final product.

| Ingredient | IIIk | IIIl | IIIm | IIIn | IIIo |
|---|---|---|---|---|---|
| Peppermint Oil | 0.15 | | | | 0.15 |
| Citrus Oil | | | | 0.15 | |
| Wintergreen Oil | | 0.1 | 0.1 | | |
| Cinnamon Oil | | | 0.05 | | |
| Citral | 0.07 | | | 0.05 | |
| Eugenyl Acetate | 0.13 | 0.13 | 0.2 | 0.15 | 0.13 |
| Cinnamic Aldehyde | | 0.07 | | | 0.07 |
| Glycerin | 5.0 | 5.0 | 5.0 | | 7.5 |
| Ethanol | 3.0 | 10.0 | 5.00 | | |
| Sucralose | 0.05 | 0.06 | 0.06 | 0.07 | 0.07 |
| Vitamin E Acetate | 0.027 | 0.042 | 0.042 | 0.06 | 0.06 |
| Cetylpyridinium Chloride | 0.045 | 0.07 | .07 | 0.010 | 0.10 |
| Water | Q.S. | Q.S. | Q.S. | Q.S. | Q.S. |

### Example IV

### Respiratory Compositions

Respiratory compositions are shown below with amounts of ingredients in weight %. Examples #1-#8 are liquid compositions made using conventional methods and may be used for example as a throat spray, rinse or gargle.

Examples #9-#16 can be made by first adding water, citric acid, sodium CMC, polyoxyl 40 stearate, and or polyethylene oxide to a clean vessel. The contents are stirred until the CMC disperses. In a second separate vessel propylene glycol, glycerin, sucrose, sucralose, flavors and flavoring agents, salivation agent and sodium benzoate are added and stirred until dissolved. The two mixtures are then combined and mixed until homogenous and then placed in a delivery device comprising the material PET.

Examples #17-#20 can be made by first adding water, citric acid, sodium CMC and poloxamer 407 to a clean vessel. The contents are stirred until the ingredients disperse. In a separate vessel the xanthan gum, guar gum and glycerin are mixed until the gums dissolve and disperse. In a third separate clean vessel the propylene glycol, sucrose, sucralose, flavors, sodium citrate and sodium benzoate are added and stirred until dissolved. The three mixtures are then combined and mixed until homogenous and then placed in a delivery device comprising the material PET.

| Ingredient | #1 | #2 | #3 | #4 |
|---|---|---|---|---|
| Carvacrol | 0.25 | 0.06 | 0.04 | |
| Eucalyptol | 0.25 | 0.30 | 0.19 | |
| Eugenyl Acetate | 0.25 | 0.18 | 0.11 | 0.15 |
| Geraniol | 0.25 | 0.24 | 0.15 | |
| Citral Diethyl Acetal | 0.25 | 0.02 | 0.01 | 0.15 |
| Polyoxyl 40 Stearate | 0.75 | 0.75 | 0.75 | 0.75 |
| Polyethylene Oxide | | 0.25 | | 0.25 |
| Sodium Carboxymethylcellulose | 0.42 | 0.45 | 0.42 | 0.45 |
| Flavor | 0.50 | 1.00 | 0.30 | 0.30 |
| Na Saccharin | 0.50 | 0.30 | | 0.20 |
| Sucralose | | 0.10 | 0.20 | |
| Sodium Benzoate | 0.10 | 0.10 | 0.10 | 0.10 |
| Benzoic Acid | 0.13 | 0.13 | 0.13 | 0.13 |
| Propylene Glycol | 15.0 | 8.0 | 15.0 | 8.0 |
| Sorbitol Solution | 15.0 | 15.0 | 15.0 | 15.0 |
| USP Water | Q.S. | Q.S. | Q.S. | Q.S. |

| Ingredient | #5 | #6 | #7 | #8 |
|---|---|---|---|---|
| Eucalyptol | 0.175 | 0.35 | 0.35 | |
| Eugenyl Acetate | 0.25 | | | 0.50 |
| Geraniol | 0.325 | 0.65 | | 0.50 |
| Citral Dimethyl Acetal | 0.25 | | 0.35 | |
| Polyoxyl 40 Stearate | 0.75 | 0.75 | 0.75 | 0.75 |
| Polyethylene Oxide | | 0.25 | | 0.25 |
| Sodium Carboxymethylcellulose | 0.42 | 0.45 | 0.42 | 0.45 |
| Flavor | 0.50 | 0.50 | 0.30 | 0.50 |
| Na Saccharin | 0.40 | 0.50 | 0.40 | 0.30 |
| Sucralose | 0.10 | | | 0.10 |
| Sodium Benzoate | 0.10 | 0.10 | 0.10 | 0.10 |
| Benzoic Acid | 0.13 | 0.13 | 0.13 | 0.13 |
| Propylene Glycol | 15.0 | 8.0 | 15.0 | 8.0 |
| Sorbitol Solution | 15.0 | 15.0 | 15.0 | 15.0 |
| USP Water | Q.S. | Q.S. | Q.S. | Q.S. |

| Ingredient | # 9 | # 10 | # 11 | # 12 | # 13 | # 14 | # 15 | # 16 |
|---|---|---|---|---|---|---|---|---|
| Peppermint Oil | 1.0 | | | | | 2.0 | | |
| Spearmint Oilr | | 1.0 | | | 3.0 | | 1.0 | 1.0 |
| Wintergreen | | | 1.0 | | | | | |
| Oil | | | | | | | | |
| Cinnamon Oil | | | | 1.0 | | | | |
| Carvacryl | | 0.20 | | 0.20 | 0.06 | 0.04 | 0.20 | 0.20 |
| Acetate | | | | | | | | |
| Eucalyptol | | 0.10 | 0.10 | 0.20 | 0.30 | 0.19 | 0.10 | 0.10 |
| Eugenyl | 0.10 | | 0.25 | 0.20 | 0.18 | 0.11 | | |
| Acetate | | | | | | | | |
| Geraniol | | 0.30 | 0.15 | 0.20 | 0.24 | 0.15 | 0.30 | 0.30 |
| Citral Diethyl | 0.40 | | 0.25 | 0.20 | 0.02 | 0.01 | | |
| Acetal | | | | | | | | |
| Propylene | 40.0 | 15.0 | 15.0 | 15.0 | 15.0 | 15.0 | 40.0 | 40.0 |
| Glycol | | | | | | | | |
| Sodium CMC | 0.45 | 0.5 | 0.5 | 0.5 | 0.5 | 0.5 | 0.45 | 0.45 |
| Citric Acid | 0.5 | 0.4 | 0.4 | 0.4 | 0.4 | 0.4 | 0.5 | 0.5 |
| Sucrose | 14 | 20.0 | 20.0 | 20.0 | 20.0 | 20.0 | 14.0 | 14.0 |
| Sucralose | 0.05 | 0.08 | 0.08 | 0.08 | 0.08 | 0.08 | 0.05 | 0.05 |
| Glycerin | 10.0 | 1.3 | 1.3 | 1.3 | 1.3 | 1.3 | 10.0 | 10.0 |
| Sorbitol 70% | | 15.0 | 15.0 | 15.0 | 15.0 | 15.0 | | |
| Solution | | | | | | | | |
| Polyoxyl 40 | | 0.6 | 0.6 | 0.6 | | 0.6 | | |
| Stearate | | | | | | | | |
| Polyethylene | | 0.2 | 0.2 | 0.2 | | 0.2 | | |
| Oxide | | | | | | | | |
| Sodium | 0.01 | 0.01 | 0.01 | 0.01 | 0.01 | 0.01 | 0.01 | 0.01 |
| Benzoate | | | | | | | | |
| Salivation | | | | | | | 0.02 | 0.10 |
| Agent¹ | | | | | | | | |
| Water | Q.S. | Q.S. | Q.S. | Q.S. | Q.S. | Q.S. | Q.S. | Q.S. |

¹Optaflow ® supplied by Symrise is an example of a salivation agent that may be used.

| Ingredient | #17 | #18 | #19 | #20 |
|---|---|---|---|---|
| Citric Acid | 0.3 | 0.3 | 0.3 | 0.3 |
| Sodium CMC | 0.3 | 0.3 | 0.3 | 0.3 |
| Propylene Glycol | 10.0 | 10.0 | 10.0 | 40.0 |
| Glycerin | | | 10.0 | 20.0 |
| Sucrose | 14.0 | 14.0 | 14.0 | 14.0 |
| Sodium Saccharin | 0.14 | 0.14 | 0.14 | 0.14 |
| Sodium Benzoate | 0.01 | 0.01 | 0.01 | 0.01 |
| Sodium Citrate Dihydrate | 0.45 | 0.45 | 0.45 | 0.45 |
| High Fructose Corn Syrup | 45.0 | 45.0 | 45.0 | 45.0 |
| Chlorpheniramine Maleate | 0.02 | | 0.02 | |
| Guaifenesin | 1.14 | 1.14 | 1.14 | |
| Dextromethorphan HBr | | | 0.67 | 0.67 |
| Peppermint Oil | 0.1 | | | |
| Citrus Oil | | 0.1 | | |
| Wintergreen Oil | | | 0.1 | |
| Cinnamon Oil | | | | 0.1 |
| Carvacrol | | | 0.03 | 0.02 |
| Eucalyptol | 0.04 | | | 0.02 |
| Eugenyl Acetate | 0.02 | 0.01 | 0.01 | 0.02 |
| Geraniol | 0.015 | | 0.01 | 0.02 |
| Citral | 0.05 | 0.04 | 0.025 | 0.02 |
| USP Water | Q.S. | Q.S. | Q.S. | Q.S. |

Examples #21-#24 can be made by first adding water, citric acid, and sodium CMC to a clean vessel. The contents are stirred until the CMC disperses. In a separate clean vessel the high fructose corn syrup, propylene glycol, respiratory ingredients (Chlorpheniramine Maleate, Guaifenesin, Dextromethorphan HBr) glycerin, menthol, sucrose, sucralose, flavors, sodium citrate and sodium benzoate are added and stirred until dissolved. The two mixtures are then combined and mixed until homogenous and then placed in a delivery device comprising the material PET.

| Ingredient | #21 | #22 | #23 | #24 |
|---|---|---|---|---|
| Peppermint Oil | 0.1 | | | |
| Citrus Oil | | 0.1 | | |
| Wintergreen Oil | | | 0.1 | |
| Cinnamon Oil | | | | 0.1 |
| Carvacryl Acetate | | | 0.03 | 0.02 |
| Eucalyptol | 0.04 | | | 0.02 |
| Eugenyl Acetate | 0.02 | 0.01 | 0.01 | 0.02 |
| Geraniol | 0.015 | | 0.01 | 0.02 |
| Citral | 0.05 | 0.04 | 0.025 | 0.02 |
| Glycerin | 20.0 | 20.0 | 20.0 | 20.0 |
| Propylene Glycol | 40.0 | 40.0 | 25.0 | 10.0 |
| Sucrose | 14.0 | 14.0 | 14.0 | 14.0 |
| Sucralose | 0.05 | 0.05 | 0.05 | 0.05 |
| Sodium Benzoate | 0.01 | 0.01 | 0.01 | 0.07 |
| Citric Acid | 0.5 | 0.5 | 0.5 | 0.5 |
| Xanthan Gum | 0.65 | | | 0.55 |
| Poloxamer 407 | | | 0.55 | |
| Guar Gum | | 0.55 | | |
| USP Water | Q.S. | Q.S. | Q.S. | Q.S. |

### Example V

### Hand Sanitizer Compositions

Hand sanitizer compositions (Va-Vd) containing the present antimicrobial blends are shown below with amounts of ingredients in weight %. These compositions are made using conventional methods.

| Ingredient | Va | Vb | Vc | Vd |
|---|---|---|---|---|
| Eugenyl Acetate | 0.67 | 0.5 | | |
| Methyl Isoeugenol | | | | 0.1 |
| Anisyl Butyrate | | | 0.2 | |
| Anisic Alcohol | 0.2 | | | |
| Dihydroanethole | | 0.4 | | |
| Cinnamyl Butyrate | | | 0.3 | |
| Cinnamic Aldehyde Dimethyl Acetal | | | | 0.3 |
| Vanillyl Isobutyrate | | | | 0.25 |
| Vanillyl Acetate | | | 0.2 | |
| Heliotropyl Propionate | | | | 0.25 |
| Ethyl Vanillyl Isobutyrate | | | 0.2 | |
| Citral | 0.33 | | | |
| Eucalyptol | | | | 0.1 |
| Geraniol | | | | 0.1 |
| Ethanol | 15.0 | 15.0 | 15.0 | 15.0 |
| L-Pyrrolidone Carboxylic Acid | 4.20 | 4.20 | 4.20 | 4.20 |
| Succinic Acid | 2.29 | 2.29 | 2.29 | 2.29 |
| Disodium Succinate Hexahydrate | 0.71 | 0.71 | 0.71 | 0.71 |
| Veragel¹ | 1.00 | 1.00 | 1.00 | 1.00 |
| Cocamidopropyl Hydroxysultaine | 0.50 | 0.50 | 0.50 | 0.50 |
| Ammonium Lauryl Sulfate | 0.90 | 0.90 | 0.90 | 0.90 |
| Sodium Olefin Sulfonate | 0.50 | 0.50 | 0.50 | 0.50 |
| Plexajel² | 1.00 | 1.00 | 1.00 | 1.00 |
| Water | Q.S. | Q.S. | Q.S. | Q.S. |

¹Aloe Vera gel (Aloe Barbadensis extract)
²Plexajel ASC supplied by Guardian Laboratories is a mixture of water, glycerin, Polyquaternium-4 and polyacrylamidomethylpropane sulfonic acid.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising one or more derivatives of parent essential oil compounds selected from aldehydes, ketones, alcohols, phenolics or acids, for use in personal care compositions at a level of at least about 0.02% by weight to provide effective antimicrobial activity.

2. A composition according to claim 1 wherein the one or more derivatives are selected from acetals of parent essential oil aldehydes or ketones; esters or ethers of parent essential oil alcohols or phenolics; esters of parent essential oil acids or mixtures thereof.

3. A composition according to claim 1 wherein the parent essential oil aldehydes or ketones are selected from citral, neral, geranial, cinnamic aldehyde, ρ-anisaldehyde, vanillin, ethyl vanillin, heliotropin, carvone, menthone or mixtures thereof .

4. A composition according to claim 1 wherein the parent essential oil alcohols or phenolics are selected from eugenol, isoeugenol, dihydroeugenol, carvacrol, carveol, geraniol, nerol, thymol, vanillyl alcohol, heliotropyl alcohol, ρ-anisyl alcohol, cinnamyl alcohol, β-ionol or mixtures thereof.

5. A composition according to claim 1 wherein the parent essential oil acids are selected from ρ-anisic acid, cinnamic acid, vanillic acid, geranic acid or mixtures thereof.

6. A composition according to claim 1 used in personal care compositions at a level of from about 0.02% to about 5.0% by weight.

7. A composition according to claim 2 wherein the one or more derivatives are blended with one or more of the parent essential oil compounds or with other essential oils or extracts.

8. A composition according to claim 7 comprising a blend of parent essential oil compounds and one or more derivatives thereof, wherein at least one of the parent essential oil compounds or derivatives thereof has an acyclic or non-ring structure and at least one other of the parent essential oil compounds or derivatives thereof has a cyclic-containing structure.

9. A composition according to claim 8 comprising two or more acyclic parent essential oil compounds or derivatives thereof.

10. A composition according to claim 8 comprising two or more cyclic-containing parent essential oil compounds or derivatives thereof.

11. A composition according to claim 8 comprising three or more acyclic parent essential oil compounds or derivatives thereof.

12. A composition according to claim 8 comprising three or more cyclic-containing parent essential oil compounds or derivatives thereof.

13. A composition according to claim 8 wherein the acyclic parent essential oil compounds are selected from citral, geraniol, or nerol and the cyclic-containing parent essential oil compounds are selected from eugenol, isoeugenol, dihydroeugenol, eucalyptol, carvacrol, thymol, carvone, cinnamic aldehyde, ρ-anisaldehyde, vanillin, ethyl vanillin, or heliotropin.

14. A composition according to claim 13 comprising one or more acyclic components selected from citral, citral dimethyl acetal, citral diethyl acetal, geraniol, geranyl acetate, geranyl propionate, or geraniol butyl ether and one or more cyclic-containing components selected from eugenyl acetate, eugenol methyl ether, isoeugenol methyl ether, cinnamic aldehyde, cinnamic aldehyde dimethyl acetal, cinnamyl butyrate, ρ-anisaldehyde, ρ-anisaldehyde propylene glycol acetal, anisyl butyrate, vanillin, vannilyl acetate, vanillyl isobutyrate, thymol, thymyl acetate, carvacrol, carvacryl acetate, carvacrol methyl ether, carvacrol ethyl ether, heliotropin, heliotropyl acetate; heliotropyl propionate, carvone or eucalyptol .

15. A composition according to claim 14 comprising citral, geraniol, eucalyptol and eugenyl acetate, each at a level of at least about 0.5% by weight.

16. A composition according to claim 14 comprising citral, geraniol, eucalyptol, carvacrol and eugenyl acetate, each at a level of at least about 0.5% by weight.

17. Personal care compositions for use on skin, hair, oral cavity, nasal passages, throat and other mucosal surfaces comprising from about 0.02% to about 5% by weight of an antimicrobial blend of parent essential oil compounds and one or more derivatives thereof, wherein at least one of the parent essential oil compounds or derivatives thereof has an acyclic or non-ring structure and at least one other parent essential oil compound or derivative thereof has a cyclic-containing structure.

18. A personal care composition according to claim 17 wherein the parent essential oil compounds or derivatives thereof in the blend are added as individual or purified chemicals.

19. An oral care composition comprising
(a) from about 0.02% to about 5% by weight of the total composition of a blend of parent essential oil compounds and one or more derivatives thereof, wherein the blend comprises one or more of acyclic components selected from citral, geraniol, nerol or derivatives thereof and one or more of cyclic-containing components selected from eugenol, isoeugenol, dihydroeugenol, eucalyptol, carvacrol, thymol, carvone, cinnamic aldehyde, ρ-anisaldehyde, vanillin, ethyl vanillin, heliotropin or derivatives thereof, and
(b) an orally-acceptable carrier,
wherein the composition provides effective antimicrobial activity against microorganisms involved in one or more undesirable oral cavity conditions selected from plaque, caries, calculus, gingivitis or breath malodor.

20. An oral care composition according to claim 19, wherein the blend comprises two or more of the acyclic parent essential oil compounds or derivatives thereof.

21. An oral care composition according to claim 19, wherein the blend comprises two or more of the cyclic-containing parent essential oil compounds or derivatives thereof.

22. An oral care composition according to claim 19 comprising from about 0.05% to 5% by weight of the total composition of the blend of parent essential oil compounds and one or more derivatives thereof.

23. An oral care composition according to claim 19 further comprising an antimicrobial active selected from cetylpyridinium chloride, zinc ion source, stannous ion source, copper ion source, a peroxide source, a chlorite ion source, chlorhexidine, triclosan, triclosan monophosphate or mixtures thereof.

24. An oral care composition according to claim 19 in a form selected from toothpaste, dentifrice, tooth gel, subgingival gel, mouthrinse, mousse, foam, denture product, mouthspray, lozenge, chewable tablet, or chewing gum.

25. An oral care mouthrinse composition according to claim 19 further comprising an antimicrobial active selected from cetyl pyridinium chloride, a zinc ion source, a stannous ion source, or mixtures thereof, and prepared as an emulsion.

26. A personal care composition according to claim 17 wherein said composition is a respiratory composition further comprising a respiratory ingredient.
